(19) United States
(12) Patent Application Publication
(10) Pub. No.: US 2010/0016732 A1
(43) Pub. Date: Jan. 21, 2010

(54) APPARATUS AND METHOD FOR NEURAL-SIGNAL CAPTURE TO DRIVE NEUROPROSTHESES OR CONTROL BODILY FUNCTION

(75) Inventors: Jonathon D. Wells, Seattle, WA (US); Mark P. Bendett, Seattle, WA (US)

Correspondence Address: Lockheed Martin Corporation;c/o Lemaire Patent Law Firm, PLLC, P.O. Box 1818, Burnsville, MN, 55337, US

(73) Assignee: LOCKHEED MARTIN CORPORATION, Bethesda, MD (US)

(21) Appl. No.: 12/505,462

(22) Filed: Jul. 17, 2009

Related U.S. Application Data
(60) Provisional application No. 61/081,732, filed on Jul. 17, 2008.
(60) Provisional application No. 61/226,661, filed on Jul. 17, 2009.

(51) Int. Cl. A61B 6/00

(52) U.S. Cl. 600/476

(57) ABSTRACT

Method and apparatus for detecting nerve activity of an animal. Some embodiments include outputting a light pulse having a wavelength onto a volume of animal tissue such that the light pulse interacts with active nerves of the tissue; measuring a light signal resulting from the interaction of the light pulse with the tissue; transmitting an electrical signal based on the measured light signal; signal-processing the electrical signal; and outputting a response signal, which can optionally be used to control a prosthetic device, stimulate another nerve, or display/ diagnose a condition. Some embodiments output a plurality of light wavelengths and/or pulses, which are optionally high-frequency intensity modulated. Some embodiments analyze DC, AC, and phase components of signals to spatially resolve locations of neural activity. Some embodiments output light pulse(s) and detect the resultant light from outside a human skull to detect neural activity of human brain tissue inside the skull.

### CROSS-REFERENCE TO RELATED APPLICATIONS

This application claims priority to U.S. Provisional Patent Application 61/081,732 (Attorney Docket 5032.044PV1) filed on Jul. 17, 2008, titled “Method and Apparatus for Neural Signal Capture to Drive Neuroprostheses or Bodily Function,” and to U.S. Provisional Patent Application 61/226,661 (Attorney Docket 5032.044PV2) filed on Jul. 17, 2009, titled “Method and Apparatus for Neural-Signal Capture to Drive Neuroprostheses or Control Bodily Function,” each of which is incorporated herein by reference in its entirety.

This invention is also related to U.S. patent application Ser. No. 11/257,793 filed Oct. 24, 2005 (Attorney Docket No. 5032.009US1) titled “Apparatus and Method for Optical Stimulation of Nerves and Other Animal Tissue,” U.S. patent application Ser. No. 11/536,639 filed Sep. 28, 2006 (Attorney Docket No. 5032.020US1) and titled “MINIATURE APPARATUS AND METHOD FOR OPTICAL STIMULATION OF NERVES AND OTHER ANIMAL TISSUE,” U.S. patent application Ser. No. 11/948,912 filed Nov. 30, 2007 (Attorney Docket No. 5032.022US1) and titled “APPARATUS AND METHOD FOR CHARACTERIZING OPTICAL SOURCES USED WITH HUMAN AND ANIMAL TISSUES,” U.S. patent application Ser. No. 11/536,642 filed Sep. 28, 2006 (Attorney Docket No. 5032.023US1) and titled “APPARATUS AND METHOD FOR STIMULATION OF NERVES AND AUTOMATED CONTROL OF SURGICAL INSTRUMENTS,” U.S. patent application Ser. No. 11/971,874 filed Jan. 9, 2008 (Attorney Docket No. 5032.026US1) and titled “METHOD AND VESTIBULAR IMPLANT USING OPTICAL STIMULATION OF NERVES,” U.S. Provisional patent application Ser. No. 12/191,301 filed Aug. 13, 2008 (Attorney Docket No. 5032.038US1) and titled “VCSEL ARRAY STIMULATOR APPARATUS AND METHOD FOR LIGHT STIMULATION OF BODILY TISSUES,” U.S. Provisional patent application Ser. No. 12/254,832 filed Oct. 20, 2008 (Attorney Docket No. 5032.039US1) and titled “SYSTEM AND METHOD FOR CONDITIONING ANIMAL TISSUE USING LASER LIGHT,” and U.S. Provisional Patent Application Ser. No. 61/015,665 filed Dec. 20, 2007 (Attorney Docket No. 5032.041PV1) and titled “LASER STIMULATION OF THE AUDITORY SYSTEM AT 1.94 μM AND MICROSECOND PULSE DURATIONS,” each of which is incorporated herein by reference in its entirety.

### FIELD OF THE INVENTION

The invention relates generally to the detection of neural activity using optics and more particularly to methods and apparatus for neural signal capture used to drive neuroprostheses or to stimulate or control bodily function.

### BACKGROUND OF THE INVENTION

Various strategies exist for detecting neural activity using light. For example, axonal swelling can be monitored based on passive movement of water across cell membranes as ions flow during an action potential (e.g., phase-sensitive optical low-coherence reflectometry).

Attached to the end of U.S. Provisional Patent Application 61/081,732 (Attorney Docket 5032.044PV1) filed on Jul. 17, 2008, titled “METHOD AND APPARATUS FOR NEURAL SIGNAL CAPTURE TO DRIVE NEUROPROSTHESES OR BODILY FUNCTION,” which is incorporated herein by reference in its entirety, are two appendices which include detailed studies of neural-signal capture. Both of these appendices are incorporated herein by reference in their entirety. Appendix A is titled “Effects of measurement method, wavelength, and source-detector distance on the fast optical signal,” and is authored by Gabriele Gratton et al. Appendix B is titled “Progress of near-infrared spectroscopy and topography for brain and muscle clinical applications,” and is authored by Martin Wolf et al. In some embodiments, the present invention uses techniques and apparatus such as described in these references in the improved invention described herein.

Another neural-activity-detection strategy involves spectroscopically analyzing chemical concentrations that mediate action potentials (AP's). Examples include analyzing increases in oxygen (O₂) consumption in the brain, monitoring concentrations of molecules that fluoresce (e.g., flavin adenine dinucleotide (FAD), nicotinamide adenine dinucleotide (NADH), etc.) using fluorescence spectroscopy, and monitoring concentrations of other molecules involved in action potential (e.g., free intracellular Ca²⁺, free neurotransmitters, etc.). Fluorescence spectroscopy can target molecules/proteins involved in the transduction or propagation of an action potential (directly or indirectly, such as blood flow). Optical coherence tomography can also be used to detect potentials (via changes in blood flow or membrane movement—leading to fast scattering changes).

There are recent descriptions of research as to the sources of human volition, which are based on electrical stimulation of the brain during surgery when the skull is open and the brain exposed. See, e.g., Haggard, “The Sources of Human Volition”, Science, 8 May 2009, Vol. 324: pp 731-733 and Desmurget et al., “Movement Intention after Parietal Cortex Stimulation in Humans,” Science, 8 May 2009, Vol. 324: pp 811-813, each of which is incorporated by reference. Desmurget et al. describe electrically stimulating patients, each being stimulated at a number of sites in the frontal, parietal and temporal regions on the exposed brain surface of the patients, and determining various particular Brodmann area (BA) sites, which, when stimulated, produced a desire to move (and/or a sensation that a movement had been accomplished) without any overt movement being produced.

Various cortical area-to-function mapping schemes exist. One mapping is based on Brodmann areas, which are regions of the cortex defined based on their cytoarchitecture, or organization of cells. Brodmann areas (BAs) were originally defined and numbered by Korbinian Brodmann in 1909. Such mapping relies on the notion that certain areas of the brain (e.g., BAs) are dedicated to particular functions, such as action execution (e.g., sending signals to muscles to move, or for speech), action inhibition, action observation, action preparation, or action motor learning. Other brain areas (e.g., BAs) are dedicated to cognition (including attention, of language, language orthography, language phonology, language semantics, language speech, language syntax, memory explicit, memory implicit, memory-working, music, reasoning, soma, space, and time), emotion (including anger, anxiety, disgust, fear, sadness), interoception (including of hunger and sexuality), perception (including audition, olfaction, somesthesis, and pain), and perception vision (including of color, motion, and shape).

U.S. Pat. No. 5,213,105 to Gratton, et al. that issued May 25, 1993 is titled “Frequency domain optical imaging using diffusion of intensity modulated radiation” and is incorporated herein by reference. This patent describes arrangements for producing images based upon diffusional-wave theory and frequency-domain analysis. A medium to be imaged is illuminated with amplitude modulated radiation, and diffusional radiation transmitted or reflected by the medium is detected at a plurality of detection locations, as by a television camera. The phase and also the amplitude demodulation of the amplitude modulated diffusional radiation are detected at each detection location. A relative phase image and also a demodulation amplitude image of the medium are then generated from respectively the detected relative phase values and the detected demodulation amplitudes of the diffusional radiation at the plurality of locations. The body is illuminated with near infrared radiation (NIR) having a wavelength between 600 and 1200 nanometers that is amplitude modulated at a frequency in the megahertz to gigahertz range, and internal images of the patient are generated for medical diagnosis.

U.S. Pat. No. 5,088,493 to Giannini, et al. that issued Feb. 18, 1992 titled “Multiple wavelength light photometer for non-invasive monitoring” is incorporated herein by reference. This patent describes a multiple wavelength light spectrophotometer for non-invasive monitoring of a body organ in vivo including: a single pulsed light source, optical fibers for transmitting to and receiving the infrared radiation from the organ, a radiation detector capable of branching received radiation into several different wavelengths, an amplifier, and a data acquisition system including a microprocessor capable of compensating for light-diffusion effects by employing a specific algorithm.

U.S. Pat. No. 5,564,417 to Chance that issued Oct. 15, 1996 titled “Pathlength corrected oximeter and the like” is incorporated herein by reference. This patent describes a path-length-corrected spectrophotometer for tissue examination that includes an oscillator for generating a carrier waveform of a selected frequency, an LED light source for generating light of a selected wavelength that is intensity modulated at the selected frequency introduced to a subject, and a photodiode detector for detecting light that has migrated in the tissue of the subject. The spectrophotometer also includes a phase detector for measuring a phase shift between the introduced and detected light, a magnitude detector for determination of light attenuation in the examined tissue, and a processor adapted to calculate the photon migration path length and determine a physiological property of the examined tissue based on the path length and on the attenuation data.

A survey paper by Peter Rolfe titled “In Vivo Near-Infrared Spectroscopy” Annu. Rev. Biomed. Eng. 2000. 02:715-54 is incorporated herein by reference. In this paper, Rolfe described various methods for determining the spatial location of structures and activities in a living person, including analysis of propagation in tissue, in vivo multivariate analysis, time-resolved spectroscopy, time-domain methods, frequency domain methods, and spatially resolved spectroscopy. Rolfe notes that light scattering has two possible forms, elastic and inelastic. With inelastic scattering, the incident energy is absorbed by the scatterer, and energy at a different wavelength is then emitted as the excited molecule falls back to one of several alternative states. This may lead to fluorescence or phosphorescence, for example. With elastic scattering, however, there is no loss of energy, but the re-emitted energy merely moves on in a different direction than that of the incoming energy. In tissues, it is possible for both elastic and inelastic scattering to take place when NIR wavelengths are used for interrogation, although most early work has been concerned with the use of elastic scattering phenomena. Early in the development of in vivo near-infrared spectroscopy (ivNIRS), it was apparent that the difference between physical (geometrical)-path length, L, and optical-path length, Lₒ, has a profound effect on calculations of chemical concentration made by using the simple Lambert-Beer law. A correction for this effect could be made if a path length factor ζ is applied to the physical-path length measurement Lₒ=ζL. Applying that analysis to a tissue sample in which scattering takes place, extending the path length from L to Lₒ by an amount that is determined from the differential-path-length factor ζ. Determination of ζ for a tissue sample allows this aspect of scatter to be accounted for, and this approach was indeed carried out by several groups (Wyatt J S, et al. 1990, “Measurement of optical path length for cerebral near infrared spectroscopy in newborn infants,” Dev. Neurosci. 12: 140-44). However, this is not the whole story, because the Lambert-Beer law must be modified appropriately to add a scattering term G, which depends on the nature of the tissues and geometry: A=log(I/I₀)=ε[C]Lζ+G. Absolute quantitative concentration cannot be obtained without knowledge of G. However, partly to overcome this difficulty, assumptions may be made that the effect of scatter remains constant, and therefore the additional scattering term can be eliminated by mathematical manipulations. This approach led to the use of multivariate analysis to determine quantitative measurement of changes in absorber concentration [ΔC] from changes in absorption ΔA. The precise wavelengths used in NIRS instruments vary somewhat, as is described below. In the earlier instruments developed by Rolfe's group, the wavelengths used were 775, 845, and 904 nm. An additional wavelength of 805 nm was also used for some experimental work. Changes in concentration of each oxygen-dependent absorber, [ΔC], can then be calculated, where the extinction coefficients for each chromophore at each of the three wavelengths are specified. This set of equations can then be used to obtain the change in concentration of each of the three absorbers. As a first approximation, it is assumed that ζ is the same for the three wavelengths. The ε_{i;j} values have been determined in vitro by using laboratory spectrophotometers (see Van Assendelft O W, “Spectrophotometry of Haemoglobin Derivatives,” Assen, The Netherlands: Vangorcum, 1970; Rea P A, Crowe J, Wickramasinghe Y, Rolfe P, “Non-invasive optical methods for the study of cerebral metabolism in the human newborn: a technique for the future?” J Med. Eng. Technol. 9(4):160-66, 1985; Wray S, Cope M, Delpy D T, Wyatt J S, Reynolds E O R, “Characterisation of the near infra-red absorption spectra of cytochrome aa₃ and haemoglobin for the non invasive monitoring of cerebral oxygenation,” Biochim. Biophys. Acta 933:184-92, 1988). The distance traveled by scattered photons between the transmitter and the receiver is longer than that traveled by unscattered photons. Approaches based on time-resolved spectroscopy (Chance B, Leigh J S, Miyake M, Smith D S, Nioka S, et al. “Comparison of time-resolved and -unresolved measurements of deoxyhemoglobin in brain,” Proc. Natl. Acad. Sci. USA 85:4971-75, 1988; Patterson M S, Chance B, Wilson B C, “Time resolved reflectance and transmittance for the noninvasive measurement of tissue optical properties,” Appl. Opt. 28:2331-36, 1989) include time-domain (TD) and frequency-domain (FD) methods. These methods were reviewed, and the theoretical basis for their operation was described thoroughly, by Arridge S R, Cope M, Delpy D T, “The theoretical basis for the determination of optical pathlengths in tissue: temporal and frequency analysis,” Phys. Med. Biol. 37(7):1531-60, 1992). Here the path-length factor introduced above, ζ, is referred to as the differential path length factor. With the TD method, a short light pulse (about 2-5 ps) is delivered to the sample and, after propagation, is detected with, for example, a streak camera (Delpy D T, Cope M, van der Zee P, Arridge S R, Wray S, Wyatt J S, “Estimation of optical pathlength through tissue from direct time of flight measurement,” Phys. Med. Biol. 33(12): 1433-42, 1988). The family of photon paths produced by scattering leads to a broadening of the pulse with the temporal point spread function (TPSF). The time tₘₐₓ at which the maximum detected intensity occurs relative to the input pulse is the mean arrival time of photons, and this may be used, together with velocity of light in vacuo (cᵥ) and tissue refractive index nₜ to calculate mean optical path length=(cᵥ/nₜ)tₘₐₓ. Use of the measurement of time gives the method its alternative name, “time-of-flight.” Although the TD method is a valuable tool for conducting basic research, the apparatus is large and expensive and not directly suited to clinical monitoring. The FD approach has the potential to overcome this problem. In FD spectroscopy, the interrogating energy is intensity modulated (IM), and the detected energy exhibits a phase shift, Φ, as compared with the modulating signal, owing to the propagation delay, as well as attenuation from absorption and scattering. The detected intensity is of the form: I=I_{dc}+I_{ac} sin(2πvt−Φ). The measurement of Φ can allow optical path length to be calculated because Lₒ=Φ cᵥ/2πvnₜ; where v is the modulating frequency, nₜ is the refractive index of the tissue, and cᵥ is the speed of light in vacuo. Because phase measurement is used in this way, the approach is also referred to as “phase modulation” (Chance B, Maris M, Sorge J, Zhang M Z, “A phase modulation system for dual wavelength difference spectroscopy of hemoglobin deoxygenation in tissues,” Proc. SPIE 1204:481-91, 1990; Weng J, Zhang M Z, Simons K, Chance B, “Measurement of biological tissue metabolism using phase modulation spectroscopic techniques,” Proc. SPIE 1431: 161-70, 1991). Measurement of tissue-absorption and scattering coefficients can also be achieved by means of a further evolution of FD methods. This approach overlaps with the concepts and techniques referred to as spatially resolved (SR) methods below. Fishkin & Gratton solved the diffusion equation by considering a homogeneous infinite medium and assuming that the modulation frequency is much smaller than the typical frequency of scattering processes (Fishkin J B, Gratton E, “Propagation of photon-density waves in strongly scattering media containing an absorbing semi-infinite plane bounded by a straight edge,” J. Opt. Soc. Am. A 10: 127-40, 1993; Fishkin J B, So P T C, Cerussi A E, Fantini S, Franceschini M A, Gratton E, “Frequency-domain method for measuring spectral properties in multiple-scattering media: methemoglobin absorption spectrum in a tissue-like phantom,” Appl. Opt. 34(7):1143-55, 1995). Spatially resolved spectroscopy (SRS) addresses the practical difficulty presented by very high absorbance during attempts to make measurements through thick tissue sections has undoubtedly led to increased efforts to gain more information from reflection, diffuse reflection, or backscatter measurements. In this mode, the input-output sites on the tissue are adjacent, and their spacing may be controlled to ensure adequate signal levels for reliable analysis. Much work has therefore been done to develop further the fundamental photon propagation relationships so that they can be applied to a variety of reflection/backscatter configurations. This is relevant to multisite measurement, sometimes called multi-distance spectroscopy, which is of growing importance. The SR method is based on solution of the diffusion approximation for a highly scattering medium. Patterson et al. (Patterson M S, Chance B, Wilson B C, “Time resolved reflectance and transmittance for the noninvasive measurement of tissue optical properties,” Appl. Opt. 28:2331-36, 1989) solved this problem with a semi-infinite half-space geometry for an input-function. See also Matcher S J, Kirkpatrick P, Nahid K, Cope M, Delpy D T, “Absolute quantification methods in tissue near infrared spectroscopy,” Proc. SPIE 2389:486-95, 1995.

A number of patents describe various aspects of NIR spectroscopy, including U.S. Pat. No. 4,768,516 by Stoddart et al. issued Sep. 6, 1988 titled “Method and apparatus for in vivo evaluation of tissue composition,” U.S. Pat. No. 4,972,331 by Chance issued Nov. 20, 1990 titled “Phase modulated spectrophotometry,” U.S. Pat. No. 5,122,974 by Chance issued Jun. 16, 1992 titled “Phase modulated spectrophotometry,” U.S. Pat. No. 5,139,025 by Lewis, et al. issued Aug. 18, 1992 titled “Method and apparatus for in vivo optical spectroscopic examination,” U.S. Pat. No. 5,187,672 by Chance, et al. issued Feb. 16, 1993 titled “Phase modulation spectroscopic system,” U.S. Pat. No. 5,213,105 by Gratton, et al. issued May 25, 1993 titled “Frequency domain optical imaging using diffusion of intensity modulated radiation,” U.S. Pat. No. 5,386,827 by Chance, et al. issued Feb. 7, 1995 titled “Quantitative and qualitative in vivo tissue examination using time resolved spectroscopy,” U.S. Pat. No. 5,402,778 by Chance issued Apr. 4, 1995 titled “Spectrophotometric examination of tissue of small dimension,” U.S. Pat. No. 6,246,892 by Chance issued Jun. 12, 2001 titled “Phase modulation spectroscopy,” U.S. Pat. No. 6,263,221 by Chance, et al. issued Jul. 17, 2001 titled “Quantitative analyses of biological tissue using phase modulation spectroscopy,” U.S. Pat. No. 6,272,367 by Chance issued Aug. 7, 2001 titled “Examination of a biological tissue using photon migration between a plurality of input and detection locations,” U.S. Pat. No. 6,542,772 by Chance issued Apr. 1, 2003 titled “Examination and imaging of biological tissue,” U.S. Pat. No. 6,564,076 by Chance issued May 13, 2003 titled “Time-resolved spectroscopic apparatus and method using streak camera,” U.S. Pat. No. 6,956,650 by Boas, et al. issued Oct. 18, 2005 titled “System and method for enabling simultaneous calibration and imaging of a medium,” U.S. Pat. No. 7,139,603 by Chance issued Nov. 21, 2006 titled “Optical techniques for examination of biological tissue,” U.S. Patent Application 20080009748 A1 by Enrico Gratton et al. published Jan. 10, 2008 titled “Method And Apparatus for the Determination of Intrinsic Spectroscopic Tumor Markers by Broadband-Frequency Domain Technology,” U.S. Patent Application 20080161697 A1 by Chance; Britton published Jul. 3, 2008 titled “Examination of subjects using photon migration with high directionality techniques,” U.S. Patent Application 20090030327 A1 by Chance; Britton published Jan. 29, 2009 titled “Optical coupler for in vivo examination of biological tissue,” and PCT Pub. No. WO/2000/025112 from International Application No. PCT/GB1999/003563 published May 4, 2000 by Peter ROLFE, titled “OPTICAL MONITORING”, each of which is incorporated herein by reference.

A number of other patents describe various aspects of NIR spectroscopy, including U.S. Pat. No. 4,840,485 by Gratton issued Jun. 20, 1989 titled “Frequency domain cross-correlation fluorometry with phase-locked loop frequency synthesizers,” U.S. Pat. No. 5,062,428 by Chance issued Nov. 5, 1991 titled “Method and device for in vivo diagnosis detecting IR emission by body organ,” U.S. Pat. No. 5,088,493 by Giannini et al. issued Feb. 18, 1992 titled “Multiple wavelength light photometer for non-invasive monitoring,” U.S. Pat. No. 5,212,386 by Gratton et al. issued May 18, 1993 titled “High speed cross-correlation frequency domain fluorometry-phosphorimetry,” U.S. Pat. No. 5,257,202 by Feddersen et al. issued Oct. 26, 1993 titled “Method and means for parallel frequency acquisition in frequency domain fluorometry,” U.S. Pat. No. 5,323,010 by Gratton et al. issued Jun. 21, 1994 titled “Time resolved optical array detectors and CCD cameras for frequency domain fluorometry and/or phosphorimetry,” U.S. Pat. No. 5,353,799 by Chance issued Oct. 11, 1994 titled “Examination of subjects using photon migration with high directionality techniques,” U.S. Pat. No. 5,553,614 by Chance issued Sep. 10, 1996 titled “Examination of biological tissue using frequency domain spectroscopy,” U.S. Pat. No. 5,664,574 by Chance issued Sep. 9, 1997 titled “System for tissue examination using directional optical radiation,” U.S. Pat. No. 5,792,051 by Chance issued Aug. 11, 1998 titled “Optical probe for non-invasive monitoring of neural activity,” U.S. Pat. No. 5,899,865 by Chance issued May 4, 1999 titled “Localization of abnormal breast tissue using time-resolved spectroscopy,” U.S. Patent Application 20020147400 A1 by Chance published Oct. 10, 2002 titled “Examination of subjects using photon migration with high directionality techniques,” U.S. Patent Application 20040073101 A1 by Chance published Apr. 15, 2004 titled “Optical techniques for examination of biological tissue,” each of which is also incorporated herein by reference.

What is needed is an apparatus and method that uses NIR to detect neural activity of a particular type and function (e.g., by deriving a spatial pattern or image of the neural activity, and in some embodiments, determine a temporal and spatial pattern), determine an intended function for that pattern (e.g., to flex the right-hand index finger), and generate a signal, image, or other data for diagnostic purposes, or to control a neuroprosthesis, to drive a neural stimulator that regenerates a compound nerve-action potential (CNAP) signal in vivo, to control a computer, speech synthesizer, or other machine or function.

### SUMMARY OF THE INVENTION

In some embodiments, the present invention provides a light-based apparatus for capturing signals indicative of neural activity. The signals are used for any of a plurality of uses, including use in prosthetic devices, nerve repair, stimulation of limbs lacking nerve connections, and the like. In some embodiments, the present invention provides a method and apparatus for detecting brain activity and particular thought patterns (e.g., for controlling prosthetic devices, stimulation of nerves to damaged limbs or organs, truth-versus-deception detection, and the like), wherein some embodiments perform such brain-activity detection non-invasively and/or from a distance (such as across a room) using person-tracking devices to maintain the laser-light source on the specific area of the brain of the person being monitored.

In some embodiments, the present invention detects nerve activity of an animal (such as activity in the brain of a human) by outputting a first light signal, which includes a light pulse having a first wavelength, onto a volume of animal tissue such that the first light signal interacts with the volume of animal tissue; detecting neural-signal activity by measuring a second light signal resulting from the interaction of the first light signal with the volume of animal tissue; transmitting an electrical signal based on the measured second light signal; processing the electrical signal; and outputting a response signal. In some embodiments, the method further includes coupling the response signal to a prosthetic device, and processing the coupled response signal in the prosthetic device to control an action by the prosthetic device. In some embodiments, the first light signal includes a plurality of wavelengths that are emitted simultaneously from one or more locations. In some embodiments, the first light signal includes a plurality of wavelengths that are emitted at different times (i.e., in a sequence). In some embodiments, the first light signal includes a plurality of pulses at a first wavelength that are emitted at different times (i.e., in a sequence). In some embodiments, the first light signal includes a plurality of pulses each having one or more wavelengths of a selected plurality of wavelengths that are emitted at different times (i.e., in a sequence). In some embodiments, the outputting of the light pulse having the first wavelength is done outside a human skull and the volume of animal tissue includes human brain tissue inside the human skull. In some embodiments, the outputting of the light pulse having the first wavelength is done at the dura of the brain inside a human skull and the volume of animal tissue includes human brain tissue inside the human skull. In some embodiments, the outputting of the light pulse having the first wavelength is done outside a human vertebra (e.g., either non-invasively from outside the skin, or as an implanted device under the skin and/or muscle but outside the vertebra) and the volume of animal tissue includes human spinal cord tissue inside the vertebra. In some embodiments, the outputting of the light pulse having the first wavelength is done through an opening formed in a human vertebra (e.g., via a light emitter embedded in a wall of the vertebra or via an optic fiber that guides light from a location at some distance from the vertebra (either from an implanted passive light receiver implanted under the skin that receives light from an emitter outside the skin, or from an implanted light-emitting device under the skin and/or muscle but outside and at some distance from the vertebra) and the volume of animal tissue includes human spinal cord tissue inside the vertebra. In some embodiments, the outputting of the light pulse having the first wavelength is done from a light emitter embedded inside a wall of the vertebra and the volume of animal tissue includes human spinal cord tissue inside the vertebra. In some embodiments, the outputting of the light pulse having the first wavelength is done from an implanted light emitter and the volume of animal tissue includes human peripheral-nerve tissue inside the human patient. In some embodiments, the outputting of the light pulse having the first wavelength is done from a light emitter external to the human patient, and after the light passes into the patient (e.g., through the skin), this initial light is received by a passive implanted light receiver (e.g., a fiber-optic array affixed to a flexible substrate, which is placed against the nerve area of interest) and the received light (i.e., still the initial light signal before interaction with the tissue of interest) is conveyed to the desired location (i.e., to the tissue of interest) within the patient via optic fibers or a fiber bundle. In some embodiments, the volume of animal tissue of interest includes human peripheral-nerve tissue and/or spinal cord tissue and/or brain tissue inside the human patient. There, the light interacts with the tissue of interest, such that the amount of light redirected to one or more detectors changes in intensity and/or the amount of delay (e.g., in some embodiments, this delay is detected by analysis of the changes to phase of the intensity modulation on the light signal). (The changes in intensity and delay are due to interaction of the light with the nerve tissue. It is thought that the interaction typically includes (1) diffusion through translucent tissue, (2) repeated scattering and/or (3) refractions due to changes in index of refraction coinciding with nerve firing or other time-varying physiological events. It is also believed changes in index of refraction cause a change in the amount of delay (such as can be measured by a change in phase of a high-frequency intensity modulation imposed on the light pulse) between the time of initial emission of the output light pulse and the time of detection of the interacted pulse.)

In some embodiments, the interaction of the first light signal with the particular nerve or brain area whose activity is being monitored causes a fluorescent emission of light having a second wavelength different than the first wavelength, and the measuring of the second light signal includes detecting light of the second wavelength.

In some embodiments, the interaction of the first light signal with the particular nerve or brain area whose activity is being monitored causes a change in scattering, reflection, birefringence or other effect on the light having the first wavelength, and the measuring of the second light signal includes detecting light of the first wavelength. In some embodiments, the first light signal is an emitted light pulse of the first wavelength and the measuring of the second light signal includes detecting light of the first wavelength during one or more time periods shortly following the emitted pulse (e.g., detecting a response waveform (e.g., the amplitude and/or phase delay) of light at the first wavelength) from one or more detection locations. In some embodiments, a mathematical transform is performed on the detected light signal from a plurality of sensors each located at a location (e.g., located at a point of a Cartesian grid or array) that permits triangulation or other location techniques to determine a location in three-dimensional space relative to the patient. For example, in some embodiments, the detector sensors are located on a grid against the scalp of the patient, and are used to determine a particular pattern of brain regions that are active (e.g., as a nerve signal is processed and propagated to different locations in the brain), wherein the pattern would normally result in sending the nerve signal to the limb being moved. For example, the pattern may start as the INTENTION for a limb movement is formed in one area of the brain (e.g., the pre-motor cortex or the presupplementary motor area, where neural activity may indicate planning the movement, but before the movement starts) and then is propagated to another region of the patient's brain where the MUSCLE/MOTOR CONTROL is effected. In a patient who is missing that limb, the detected nerve signal can then be used to control a prosthetic limb, while in another patient who has nerve damage in nerves to a limb or organ, the detected nerve signal can then be used to control a nerve-stimulation device that causes a nerve stimulation beyond the nerve-damage area in order to obtain control of the limb or organ.

In some embodiments, the first light signal is an emitted light pulse of the first wavelength and the measuring of the second light signal includes detecting light of a second wavelength, which is different than the first wavelength, during one or more time periods shortly following the emitted pulse (e.g., detecting a response waveform of light at the second wavelength, e.g., a wavelength that is a fluorescent re-emission of light that was absorbed by some region of the nerve or surrounding tissue). In some embodiments, the first light signal is an emitted light pulse at each of a first plurality of wavelengths and the measuring of the second light signal includes detecting light at each respective one of the first plurality of wavelengths, which are the same respective wavelengths that were originally emitted, during one or more time periods shortly following the emitted pulse (e.g., detecting a response waveform of light at the second wavelength). In some embodiments, the detected light of the second wavelength is indicative of a brain activity.

### BRIEF DESCRIPTION OF THE DRAWINGS

FIG. 1A is a block diagram of neural-signal-capture system 101 according to some embodiments of the present invention.

FIG. 1B is a block diagram of neural-signal-capture system 102 according to some embodiments of the present invention.

FIG. 1C is a block diagram of single-laser vertical cavity surface emitting laser (VCSEL) source 103 according to some embodiments of the present invention.

FIG. 1D is a block diagram of one-dimensional VCSEL source linear array 104 according to some embodiments of the present invention.

FIG. 1E is a block diagram of two-dimensional VCSEL source array 105 according to some embodiments of the present invention.

FIG. 1F is a block diagram of two-dimensional VCSEL source/detector array 106 according to some embodiments of the present invention.

FIG. 1G is a block diagram of flex-cuff linear VCSEL source/detector array 107 according to some embodiments of the present invention.

FIG. 1H is a block diagram of neural-signal-capture system 108 according to some embodiments of the present invention.

FIG. 2A is a block diagram of neural-signal-capture system 201 according to some embodiments of the present invention.

FIG. 2B is a block diagram of neural-signal-capture system 202 according to some embodiments of the present invention.

FIG. 2C is a block diagram of neural-signal-capture system 203 according to some embodiments of the present invention.

FIG. 2D is a block diagram of neural-signal-capture system 204 according to some embodiments of the present invention.

FIG. 2E is a block diagram of neural-signal-capture system 205 according to some embodiments of the present invention.

FIG. 2F is a block diagram of neural-signal-capture system 206 according to some embodiments of the present invention.

FIG. 3A is a block diagram of neural-signal-capture system 301 that uses a square-pulse light signal according to some embodiments of the present invention.

FIG. 3B is a block diagram of neural-signal-capture system 302 that uses a plurality of simultaneous intensity-modulated-pulse light signals according to some embodiments.

FIG. 3C is a block diagram of neural-signal-capture system 303 that uses a plurality of sequential intensity-modulated-pulse light signals according to some embodiments.

FIG. 3D is a block diagram of neural-signal-capture system 304 that uses a plurality of rigid-unit portions, each having a plurality of VCSELs and a plurality of circumferential detectors, that are interconnected using flex circuitry according to some embodiments.

FIG. 3E is a plan-view block diagram of rigid unit 305 having a plurality of VCSELs and a plurality of circumferential detectors according to some embodiments.

FIG. 3F is a cross-section-view block diagram of VCSEL/detector 306 having one VCSEL and a plurality of circumferential detectors according to some embodiments.

FIG. 3G is a block diagram of neural-signal-capture system 307 that uses one or more intensity-modulated-pulse light signals and a plurality of detectors according to some embodiments of the present invention.

### DETAILED DESCRIPTION

Although the following detailed description contains many specifics for the purpose of illustration, a person of ordinary skill in the art will appreciate that many variations and alterations to the following details are within the scope of the invention. Accordingly, the following preferred embodiments of the invention are set forth without any loss of generality to, and without imposing limitations upon the claimed invention.

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention.

The leading digit(s) of reference numbers appearing in the Figures generally corresponds to the Figure number in which that component is first introduced, such that the same reference number is used throughout to refer to an identical component that appears in a plurality of figures. Signals and connections may be referred to by the same reference number or label, and the actual meaning will be clear from its use in the context of the description.

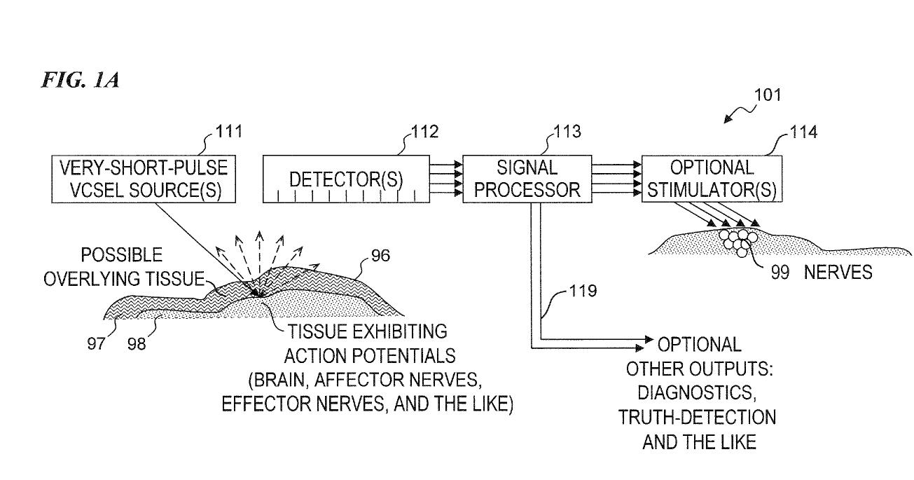

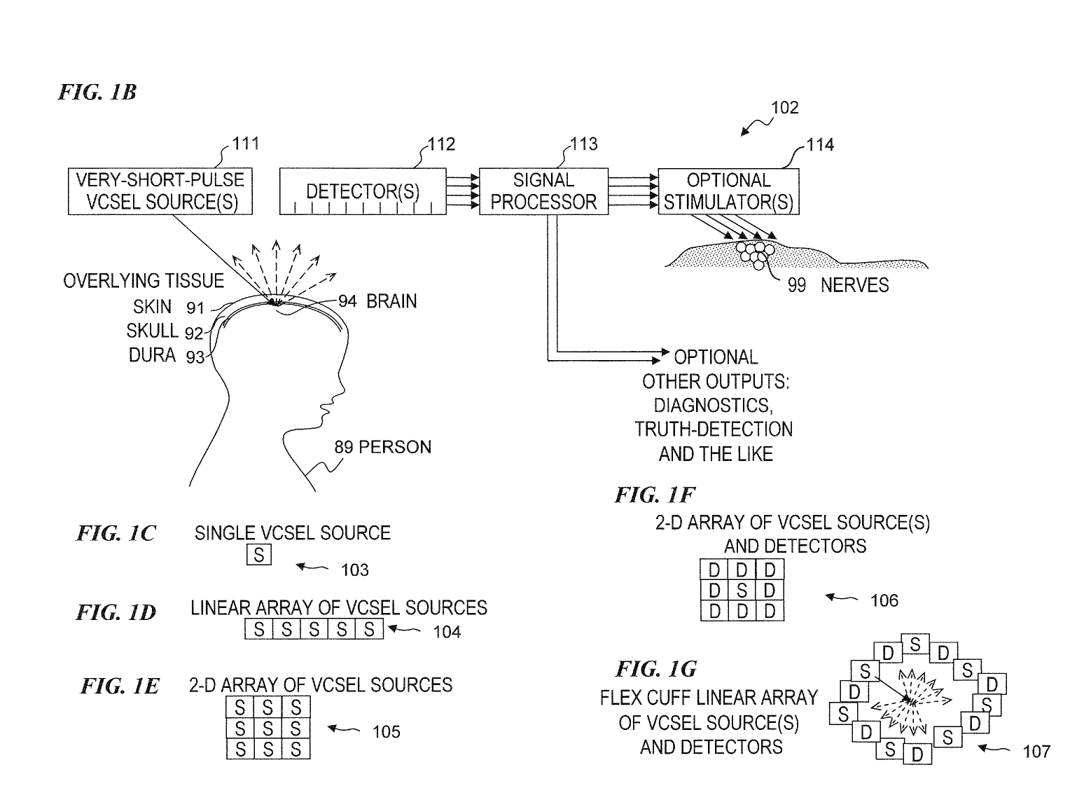

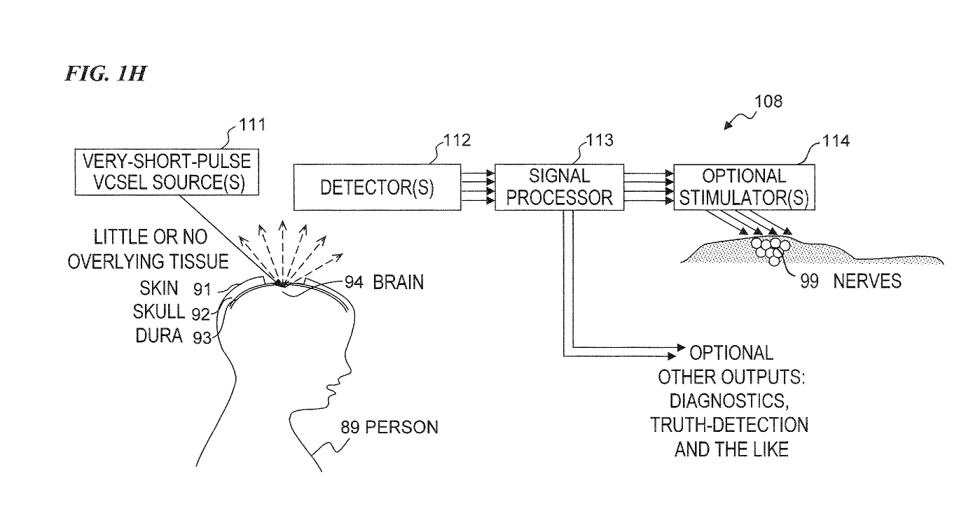

FIG. 1A is a block diagram of neural-signal-capture system 101 according to some embodiments of the present invention. In some embodiments, system 101 includes one or more light sources 111 (such as a VCSEL (vertical-cavity surface-emitting laser), VCSEL array, point-source LED (light-emitting diode) array, and the like). In some embodiments, the light sources 111 emit light toward tissue volume 96 (which may include overlying tissue 97 (e.g., skin, muscle and/or bone) and the tissue of interest 98). The scattered or reflected light returns and is detected by detectors 112, which generate electrical signals that are analyzed by signal processor 113. The signal processor 113 outputs one or more control signals 119 (e.g., to control a nerve stimulator 114 that optically and/or electrically stimulates nerves 99 of the patient, thereby possibly bypassing areas of the patient's nerve or brain damage). In some embodiments, the control signals are coupled to a display that displays the spatial and temporal patterns of neural activity. In some embodiments, the control signals are coupled to a diagnosis apparatus that performs an analysis (e.g., a medical diagnosis or truth-versus-deception detection) of the spatial and temporal patterns of neural activity. In some embodiments, the control signals are coupled to a prosthesis (e.g., a neuroprosthesis, robotic arm or leg, or the like) that performs some function for the patient.

In some embodiments, the light sources 111 emit light in the wavelength range of 680 nm to 850 nm (in some embodiments, wavelengths of about 830 nm are used to improve the signal-to-noise (S/N) ratio; however, other embodiments use one or more different wavelengths in the range 800 nm to 850 nm. In some embodiments, wavelengths of 680 nm, 750 nm, 830 nm, 775 nm, 845 nm, 904 nm and/or 805 nm are used. In some embodiments, very short substantially square pulse sources are used (outputting pulses that are shorter than 1 nanosecond (ns)), while in other embodiments, pulses having a duration in the range of 1 ns to 10 ns or even to 100 ns are used. In some embodiments, the pulses are also intensity modulated with a high-frequency sine wave (e.g., using a modulation frequency of 1 GHz, a 10-ns pulse will have ten cycles of the one-GHz intensity modulation, while a 100-ns pulse will have one hundred cycles of the one-GHz intensity modulation. In other embodiments, the present invention uses square pulses having pulse durations in the range of less than about 1 picosecond (ps) to about 1 millisecond (ms) (e.g., in some embodiments, the duration of emitted pulses is in a range of about 1 to 10 ps; in other embodiments, the duration of emitted pulses is in a range of about 1 to 1000 femtoseconds (fs), inclusive; a range of about 10 to about 100 ps, inclusive; a range of about 100 to about 200 ps, inclusive; a range of about 200 to about 500 ps, inclusive; a range of about 500 to about 1000 ps, inclusive; a range of about 1 to about 2 ns, inclusive; a range of about 2 to about 5 ns, inclusive; a range of about 5 to about 10 ns, inclusive; a range of about 10 to about 20 ns, inclusive; a range of about 20 to about 50 ns, inclusive; a range of about 50 to about 100 ns, inclusive; a range of about 100 to about 200 ns, inclusive; a range of about 200 to about 500 ns, inclusive; a range of about 500 to about 1000 ns, inclusive; a range of about 1 to about 2 μs (microseconds), inclusive; a range of about 2 to about 5 μs, inclusive; a range of about 5 to about 10 μs, inclusive; a range of about 10 to about 20 μs, inclusive; a range of about 20 to about 50 μs, inclusive; a range of about 50 to about 100 μs, inclusive; a range of about 100 to about 200 μs, inclusive; range of about 200 to about 500 μs, inclusive; and/or a range of about 500 to about 1000 μs, inclusive.

In some embodiments, suitably long pulses are also intensity modulated with a modulation frequency of between about 50 MHz or less to about 1 GHz or more. For example, some embodiments output one or more pulses, each modulated with a frequency selected from the set consisting of 10 MHz, 20 MHz, 30 MHz, 40 MHz, 50 MHz, 60 MHz, 70 MHz, 80 MHz, 90 MHz, 100 MHz, 110 MHz, 120 MHz, 130 MHz, 140 MHz, 150 MHz, 160 MHz, 170 MHz, 180 MHz, 190 MHz, 200 MHz, 210 MHz, 220 MHz, 230 MHz, 240 MHz, 250 MHz, 260 MHz, 270 MHz, 280 MHz, 290 MHz, 300 MHz, 310 MHz, 320 MHz, 330 MHz, 340 MHz, 350 MHz, 360 MHz, 370 MHz, 380 MHz, 390 MHz, 400 MHz, 410 MHz, 420 MHz, 430 MHz, 440 MHz, 450 MHz, 460 MHz, 470 MHz, 480 MHz, 490 MHz, 500 MHz, 510 MHz, 520 MHz, 530 MHz, 540 MHz, 550 MHz, 560 MHz, 570 MHz, 580 MHz, 590 MHz, 600 MHz, 610 MHz, 620 MHz, 630 MHz, 640 MHz, 650 MHz, 660 MHz, 670 MHz, 680 MHz, 690 MHz, 700 MHz, 710 MHz, 720 MHz, 730 MHz, 740 MHz, 750 MHz, 760 MHz, 770 MHz, 780 MHz, 790 MHz, 800 MHz, 810 MHz, 820 MHz, 830 MHz, 840 MHz, 850 MHz, 860 MHz, 870 MHz, 880 MHz, 890 MHz, 900 MHz, 910 MHz, 920 MHz, 930 MHz, 940 MHz, 950 MHz, 960 MHz, 970 MHz, 980 MHz, 990 MHz, 1000 MHz, 1100 MHz, 1200 MHz, 1300 MHz, 1400 MHz, 1500 MHz, 1600 MHz, 1700 MHz, 1800 MHz, 1900 MHz, 2000 MHz, 2100 MHz, 2200 MHz, 2300 MHz, 2400 MHz, 2500 MHz, 2600 MHz, 2700 MHz, 2800 MHz, 2900 MHz, 3000 MHz, 3100 MHz, 3200 MHz, 3300 MHz, 3400 MHz, 3500 MHz, 3600 MHz, 3700 MHz, 3800 MHz, 3900 MHz, 4000 MHz, 4100 MHz, 4200 MHz, 4300 MHz, 4400 MHz, 4500 MHz, 4600 MHz, 4700 MHz, 4800 MHz, 4900 MHz, 5000 MHz. In some embodiments, modulation frequencies above 5 GHz (e.g., within the range of 5 GHz to 100 GHz) are used.

Water has an index of refraction of about 1.33, while air has an index of refraction of about 1.0003. Light in air travels about 30 centimeters per nanosecond. Light in water travels about 22.6 centimeters per nanosecond, which is 0.226 millimeters (mm) per picosecond (the inverse being about 4.42 picoseconds per mm). If the light is reflected or scattered substantially directly back to a detector next to the light emitter, a resolution of about 10 picoseconds should locate the reflecting region within about 2.26 mm for the round trip, which should give a depth (one-way distance) resolution of about 1.1 mm. In some preferred embodiments, pulses having a duration in the range of 10 ps to 20 ps are used, which may result in a depth resolution (i.e., a precision of location determination) of about 1 to 2 mm measuring from a center of the emitted pulse to the center of the reflected pulse. In some embodiments, a measurement to a leading or trailing edge of the pulse is used, which may provide much finer resolution, e.g., submillimeter.

Since a compound nerve action potential (CNAP) pulse will have a duration of about 0.25 to 0.5 milliseconds, the present invention can transmit a large number of light pulses across the period in which a single CNAP pulse is active at a given location. For example, in some embodiments, pulses are emitted every 100 nanoseconds (10 million pulses per second), such that 2500 to 5000 pulses can be emitted and detected during a single CNAP pulse (with a 10-ps pulse duration, the duty cycle of the light pulses (e.g., laser pulses) in such a system would be about 0.0001). In other embodiments, pulses are emitted at other intervals, such as every 1 microsecond (1 million pulses per second), such that 250 to 500 pulses can be emitted and detected during a single CNAP pulse, or such as every 10 microseconds (100 thousand pulses per second), such that 25 to 50 pulses can be emitted and detected during a single CNAP pulse, or even such as every 100 microseconds (10 thousand pulses per second), such that 2 to 5 pulses can be emitted and detected during a single CNAP pulse.

In some embodiments, the distance to the active neural tissue (the tissue that causes a change in interaction with the light) is determined by starting a timing pulse when the light is emitted or launched toward the tissue volume of interest, and terminating the timing pulse with the reflected signal is detected, such that the duration of the timing pulse is proportional to the distance to the region that reflected the light pulse. In some embodiments, pulse durations of 1 nanosecond, 2 nanoseconds, 5 nanoseconds or 10 nanoseconds are used, wherein the leading edge of the emitted pulse to the leading edge of the reflected pulse are the triggers for the start and end of the timing pulse, respectively. For example, if the leading (or trailing) edge of a 5-nanosecond emitted light pulse starts the timing pulse and the leading (or trailing) edge of the reflected pulse stops the timing pulse, and the anomaly that retro-reflects the pulse (reflects the pulse at substantially 180 degrees, straight back at the emitter) is about 10 mm deep in tissue that has an index of refraction approximately the same as water, the timing pulse would have a duration of about 88.5 picoseconds ((the round-trip distance of 20 mm) times (the speed of light in water of 4.425 ps/mm)=88.5 ps). If the anomaly were about 11 mm deep in the same tissue, the timing pulse would have a duration of about 97.3 picoseconds. Thus a measurement of the time-of-flight timing pulse to within about plus-or-minus 8.8 picoseconds will yield a depth resolution of about plus-or-minus 1 mm. Accordingly, in some embodiments, when using relatively long pulses (e.g., 1 to 20 nanoseconds pulse duration), it is important to have a relatively fast rise time (if using the leading edge of the pulse) or a relatively fast fall time (if using the trailing edge of the pulse) in order to accurately determine the depth to (or three-dimensional location of) the active neural tissue by such time-of-flight measurements.

In some such embodiments, time-of-flight measurements are used to detect the distance to the particular nerve or brain area whose activity is being monitored. For example, in some embodiments, time-of-flight measurements measure the time between when the pulse is emitted (e.g., the time of this event could be measured from the start of the pulse, when the pulse's leading edge first reaches 1/e or ½ of the maximum intensity, the middle of the pulse (if the pulse is relatively short, e.g., 5 to 10 picoseconds for a resolution of about 1 mm) or the end (trailing edge) of the pulse) until the corresponding feature (e.g., leading or trailing edge) of the pulse that is reflected or scattered due to nerve activity is detected.

In some embodiments, a tissue phantom (simulated tissue material having one or more reflective or scattering anomalies at known locations) is used to help calibrate the time-of-flight-to-distance calculation. In some such embodiments, several different nerves at different locations are substantially simultaneously monitored by emitting short pulses at different times, and different detectors 112 detecting different scattering patterns are processed with processor 113 using techniques similar to those used for processing x-ray CAT scans or MRI scans. In some embodiments, nerve stimulators 114 are used to stimulate nerves that may have been severed or otherwise damaged. In some embodiments, other outputs are generated by processor 113, such as outputting diagnostics, driving neuro-modulation devices or neuroprostheses, truth-versus-deception detection, and the like.

In some embodiments, the source-to-detector separation is used to probe various depths of tissue and relates to the spatial precision of our signal capture. For example, aiming the emitter(s) to transmit the light pulse at a 45-degree angle to the external skin surface and against the skull, and spacing the detectors about 2.8 cm away, the detectors pointing back at about a 45-degree angle to the external skin surface, a volume of tissue about 1.4 cm deep half way between the emitters and detectors can be monitored. Also, in some embodiments, the angles of orientation of both source and detector are adjusted to empirically determine and maximize the signal captured from the detected light.

The inventors recognize that various areas of the brain (such as the cortex), spinal cord, and peripheral nerves are spatially organized to a specific function. For example, the motor control of the foot starts in a specific area of the brain (for the intent to move) then goes to another area of the brain (the motor-control initiation) and then is transmitted within a specific nerve-bundle location within the spinal cord. In some embodiments, by placing a cuff of sources and detectors around the spinal cord at a suitable vertebra along the patient's spinal column and measuring (through reflection and/or transmission) the light signal, some embodiments use signal processing to get information about a very small volume or cross section of neural tissue—specifically, the amplitude, position, and timing of the signal within the spinal cord. This yields information of the functional intent of the neuron or group of neurons firing which can be used as a diagnostic tool or to drive a closed-loop prosthetic device (for example, to bypass an area of nerve damage lower in the spinal cord). Similarly, if the nerve damage is quite high along the spinal cord, making it difficult or impossible to detect nerve activity in the spinal cord, some embodiments detect brain activity in the motor-control area for the specific muscle movement, or even detect brain activity in the intention-forming areas of the brain (to detect when the patient is forming the intent for a particular motion even before that intent is transferred to the motor-control area) if brain activity in the motor-control area is damaged or for some other reason difficult or impossible to accurately monitor.

FIG. 1B is a block diagram of neural-signal-capture system 102 according to some embodiments of the present invention. In some embodiments, system 102 transmits the light pulses from light source 111 through overlying tissue such as skin 91, skull bone 92, and dura 93 in order to illuminate excitable neuronal tissue 94 (such as the brain, spinal cord, spinal roots, peripheral nerves and/or sensory nerves) non-invasively. In other embodiments, an implanted device is used, wherein the system 102 is configured to be implanted within the patient to perform the nerve-activity measurement and the resulting control function. In some embodiments, detectors 112 are arrayed around a room and surreptitiously used to monitor a subject person 89. The use of infra-red illumination light sources 111 such as VCSEL lasers prevents the subject person 89 from knowing she or he is being monitored.

FIG. 1C is a block diagram of single-laser vertical cavity surface emitting laser (VCSEL) source 103 according to some embodiments of the present invention. Source 103 can be a semiconductor VCSEL, but in other embodiments, point-source diodes, LEDs, diode-laser-pumped fiber-based lasers (wherein one or more rare-earth species are used as a dopant in the optical fiber, wavelength-converted (e.g., a frequency-doubled erbium laser, wherein the erbium laser emits at about 1550 nm (which does not penetrate human tissue to any great extent) and this light is frequency-doubled to about 775 nm (which will penetrate human tissue fairly well))), or other lasers are used.

FIG. 1D is a block diagram of one-dimensional VCSEL source linear array 104 according to some embodiments of the present invention. In some embodiments, array 104 consists of an integrated linear array of VCSELs.

FIG. 1E is a block diagram of two-dimensional VCSEL source array 105 according to some embodiments of the present invention. In some embodiments, array 105 includes laser sources with one specific wavelength. In other embodiments, array 105 includes laser sources having a plurality of different wavelengths. In some embodiments, a single pulse duration and a single pulse-repetition-rate (PRR) frequency are used, while in other embodiments, a plurality of different pulse durations and/or PRR frequencies are used. In some embodiments, array 105 consists of an integrated two-dimensional array of VCSELs.

FIG. 1F is a block diagram of two-dimensional VCSEL source/detector array 106 according to some embodiments of the present invention. In some embodiments, a plurality of different detector types is used, wherein each type is configured to be sensitive to different wavelengths. In some embodiments, a plurality of otherwise substantially similar detectors are coated with wavelength-selective filter coatings or Fabry-Pérot interferometers (e.g., either all tuned to one specific wavelength or, in other embodiments, tuned to a plurality of different wavelengths) to only accept one or more specific wavelengths—thus boosting signal-to-noise ratios. In some embodiments, system 106 is substantially similar to array 105, with the exception that some of the sources have been replaced by detectors. In some embodiments, a VCSEL-type device can be used as a detector by appropriate changes to the biasing circuitry.

FIG. 1G is a block diagram of flex-cuff linear VCSEL source/detector array 107 according to some embodiments of the present invention.

FIG. 1H is a block diagram of neural signal capture system 108 according to some embodiments of the present invention. In some embodiments, system 108 is substantially similar to system 102 of FIG. 1B, with the exception that an opening in the skull has been created to obtain finer resolution in the sensing of different nerve areas.

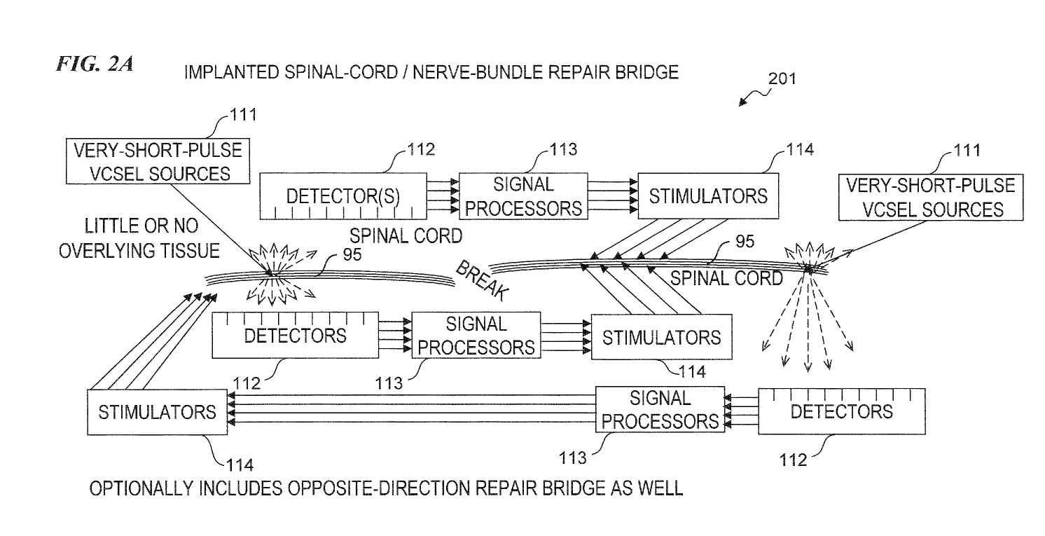

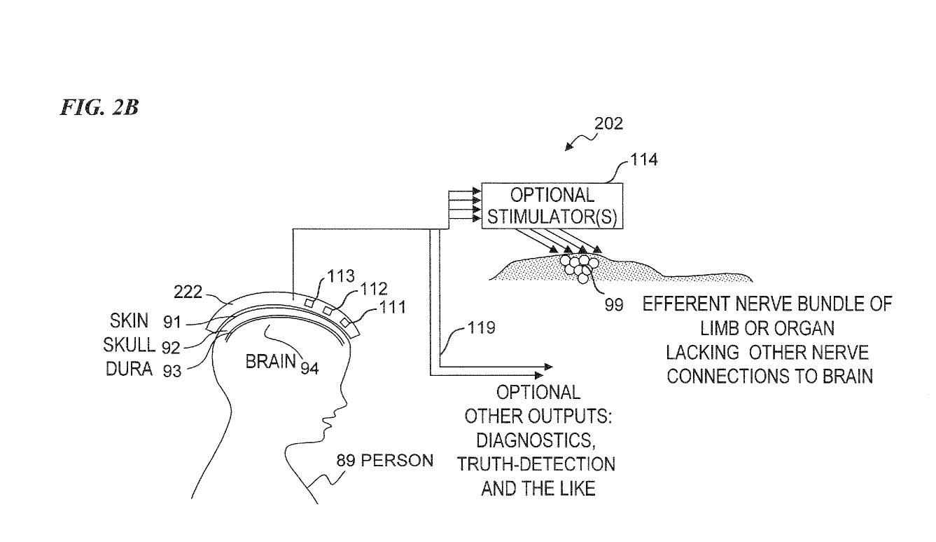

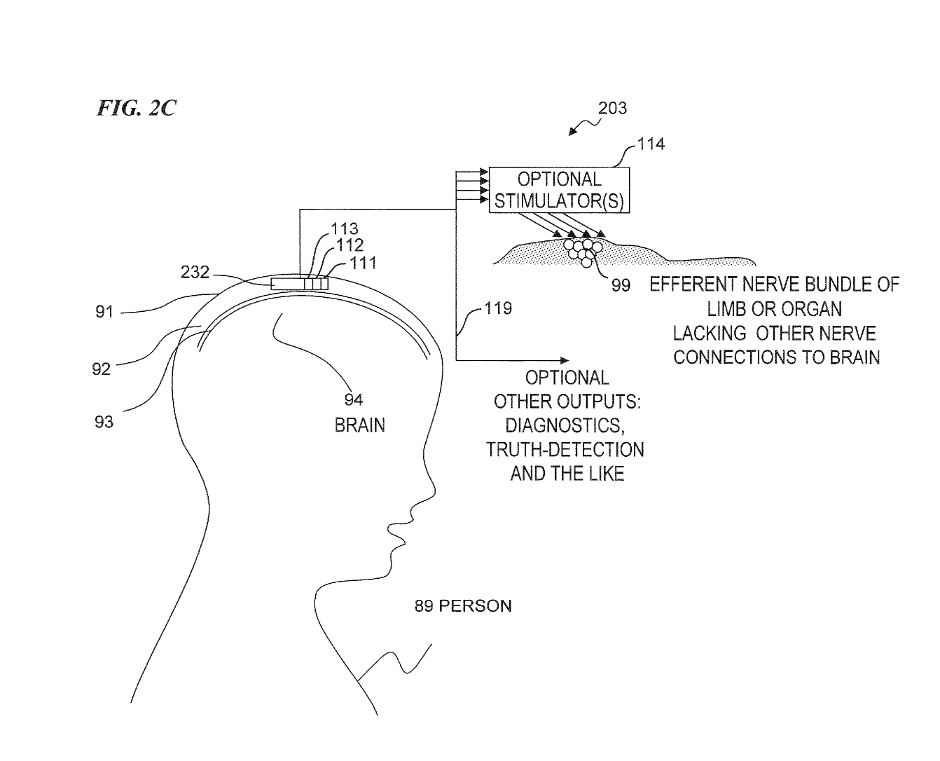

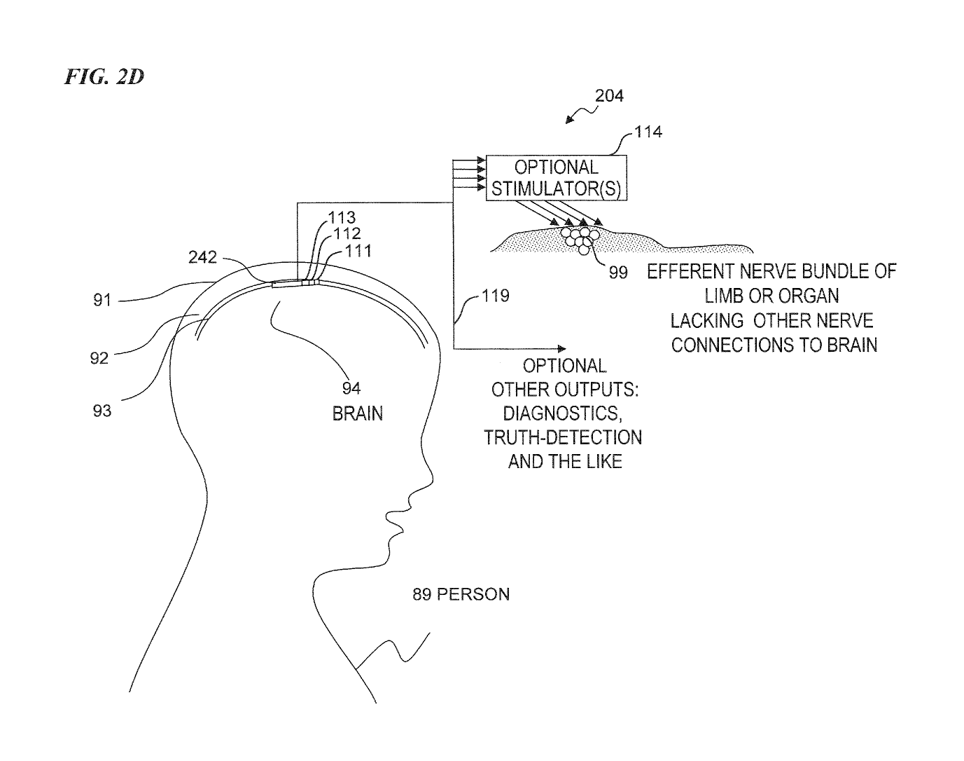

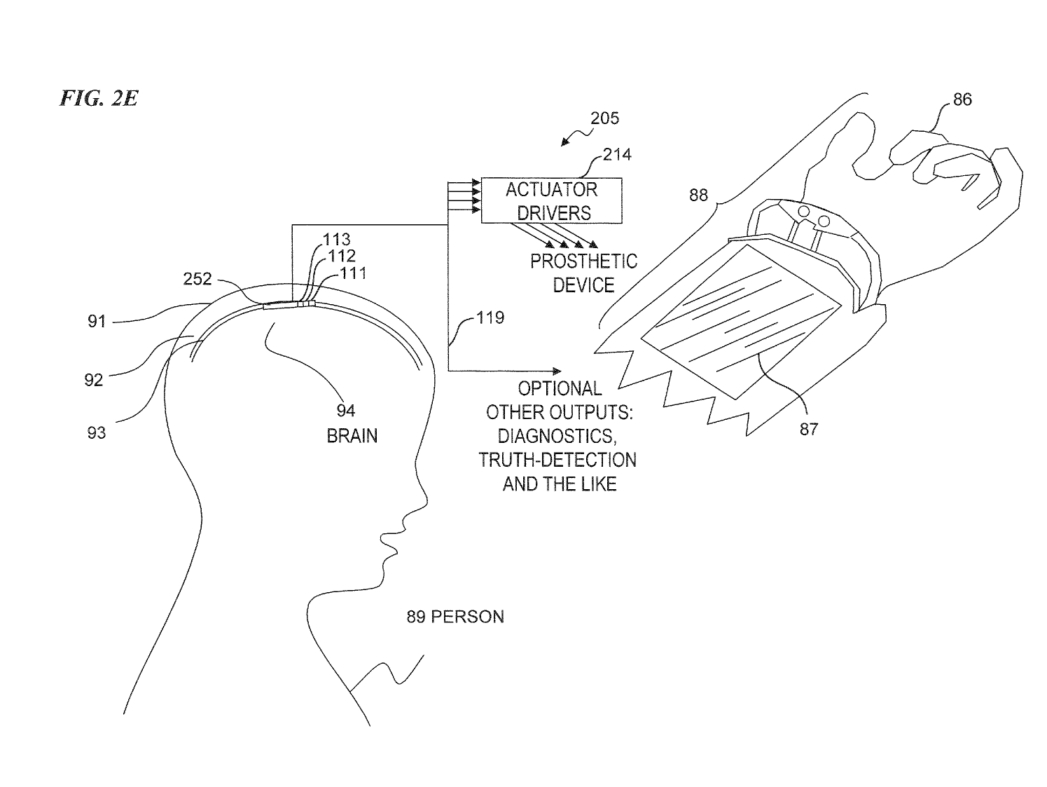

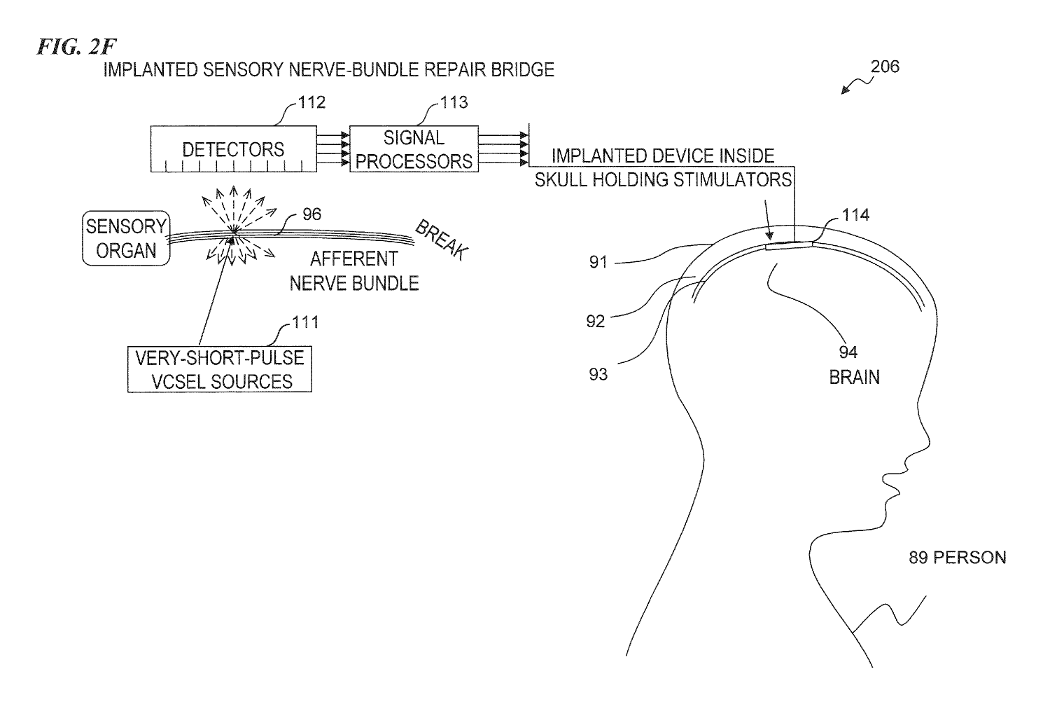

FIG. 2A is a block diagram of neural-signal-capture system 201 according to some embodiments of the present invention. In some embodiments, system 201 is used to detect specific nerves in a nerve bundle of spinal cord 95. In other embodiments, system 201 is used to detect specific neural activity in nerves in a nerve bundle of peripheral nerves. In some embodiments, detectors 112 and light source 111 are arranged in a flexible cuff surrounding some or all of spinal cord 95, in order to detect nerve signals on one side of a break in the spinal cord and to create stimulation signals from device 114 to a portion of the spinal cord 95 on the opposite side of the break. In some embodiments, system 201 includes a series of devices that sense nerve signals going the opposite direction and recreating stimulation back on the original side of the break.

FIG. 2B is a block diagram of neural-signal-capture system 202 according to some embodiments of the present invention. In some embodiments, system 202 includes a non-invasive cap holding light sources 111, detectors 112, and processors 113. In some embodiments, system 202 is used to detect neural activity in one or more specific brain areas of a cerebral cortex or other brain area of brain 94. In other embodiments, system 202 is used to detect specific nerves in one or more other brain areas. In some embodiments, detectors 112 and source(s) 111 are arranged in a non-invasive opaque cap 222 (which, in some embodiments, holds one or more very-short-pulse VCSEL sources 111, detectors 112, and signal processors 113), which, when operating, surrounds some or all of the head of person 89, in order to detect neural activity in an area of the brain (e.g., in a case where the person 89 has some brain damage or damage in the spinal cord such that she or he can no longer control some motor or speech function). System 202 creates stimulation signals from nerve-stimulation device 114 to nerves 99 (e.g., in the case shown here, one or more efferent nerves of a limb or organ lacking effective connections to the brain) in a portion of the spinal cord 95 or peripheral nervous system closer to the muscles or organ to be controlled. In some embodiments, system 202 also includes one or more devices that sense nerve signals going the opposite direction and recreating brain-detected sensations in a certain area of the brain 94 using nerve stimulation of either nerves in the spinal cord or brain.

FIG. 2C is a block diagram of neural-signal-capture system 203 according to some embodiments of the present invention. In some embodiments, system 203 includes an implanted device 232 (which, in some embodiments, holds light sources (e.g., VCSEL arrays) 111, detectors 112, and signal processors 113) that has been embedded in the skull bone 92. In some embodiments, other aspects of system 203 are as described above for FIG. 2A. In some embodiments, system 203 has the advantages of being more stable (less movement relative to the brain), while being less invasive than system 204 described below. System 203 also has the advantages of having less tissue to go through (relative to system 202 described above) to reach the areas of the brain that are being monitored.

FIG. 2D is a block diagram of neural-signal-capture system 204 according to some embodiments of the present invention. In some embodiments, system 204 includes an implanted device 242 (holding light sources 111, detectors 112, and processors 113) that has been implanted between the skull bone 92 and the brain 94. In some embodiments, other aspects of system 203 are as described above for FIG. 2A. In some embodiments, system 204 has the advantages of being perhaps even more stable (less movement relative to the brain), although being more invasive than systems 202 and 203 described above. System 204 also has the advantages of having much less tissue to go through (relative to system 202 or system 203 described above) to reach the areas of the brain that are being monitored.

FIG. 2E is a block diagram of neural-signal-capture system 205 according to some embodiments of the present invention. In some embodiments, system 205 is substantially similar to system 204 of FIG. 2D with the exception that, in some embodiments, the detected brain patterns are further analyzed in device 214 and used to drive actuator drives 87 that drive movement in appendages (e.g., fingers 86) and other functions of prosthetic device 88 for person 89. Implanted device 252 is otherwise similar to device 242 described for FIG. 2D above.

FIG. 2F is a block diagram of neural-signal-capture system 206 according to some embodiments of the present invention. In some embodiments, system 206 is used in the opposite direction of previous conventional devices, such as those described in the related patent applications and patents listed at the beginning of this application, in that sensory nerve signals are provided and stimulation signals conveying the sensory device are sent to the brain 94. For example, in some embodiments, the light sources 111 emit light toward tissue volume 96 (which, as described above in FIG. 1A, may include overlying tissue 97 (e.g., skin, muscle and/or bone) and the tissue of interest 98 (in this case, an afferent nerve bundle)). The scattered or reflected light returns or is transmitted generally through the tissue volume 96 and is detected by detectors 112, which generate electrical signals that are analyzed by signal processor 113. The signal processor 113 outputs one or more control signals (e.g., to control a nerve stimulator 114 that optically and/or electrically stimulates brain 94 of the person 89, thereby, if the person is affected by such problems, bypassing areas of the patient's nerve damage or brain damage) based on the actual senses of person 89 whose afferent nerves (within tissue 96) are monitored.

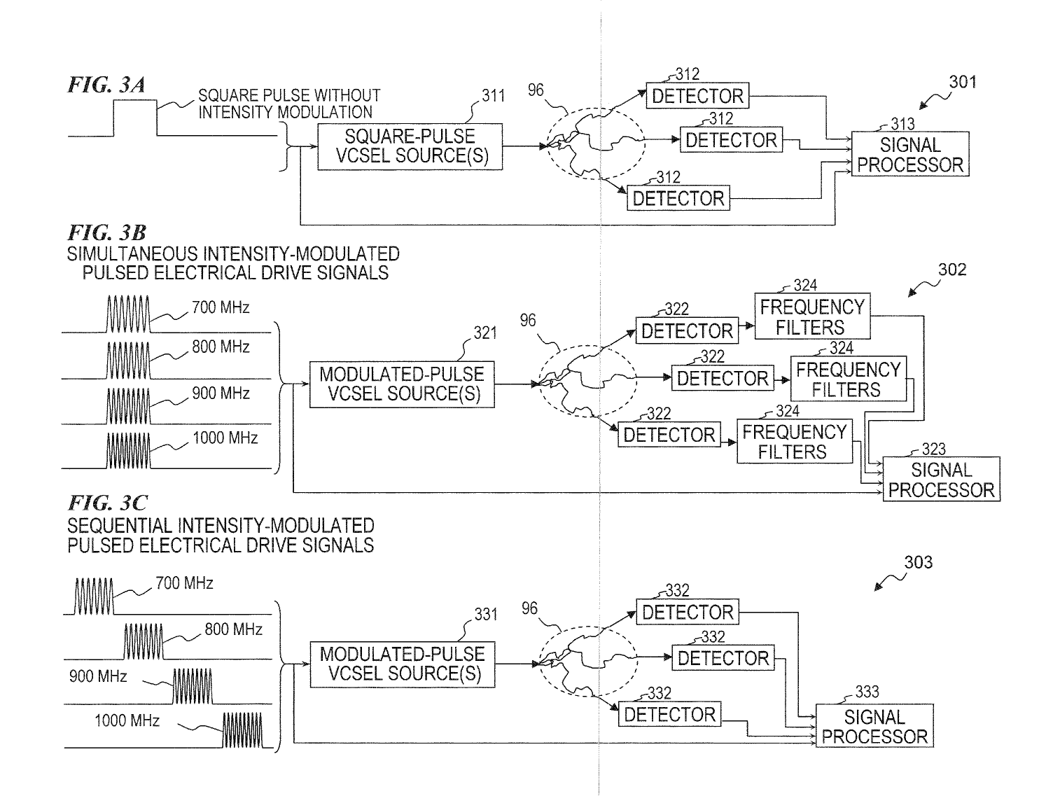

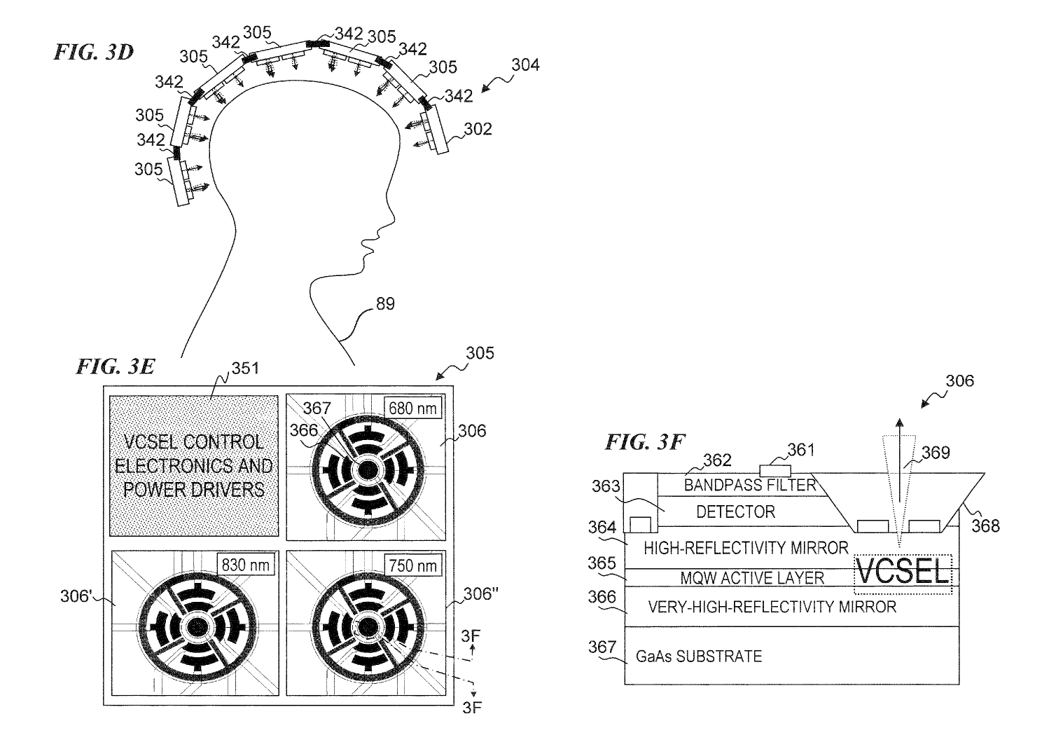

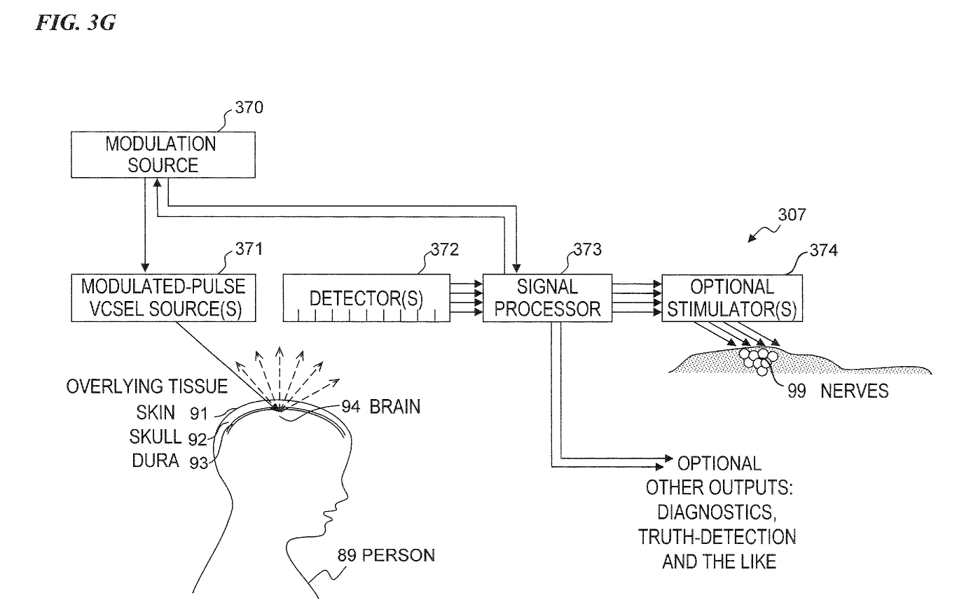

FIG. 3A is a block diagram of neural-signal-capture system 301 that uses a square-pulse light signal (e.g., a pulse that is not modulated with a higher-frequency sine wave such as are described in FIG. 3B and FIG. 3C) according to some embodiments of the present invention. In some embodiments, system 301 provides an electrical signal having the square-pulse shape as shown (amplitude in the vertical direction versus time in the horizontal direction), and applies the electrical pulse to one or more VCSEL sources 311 (which generate a light pulse having a light intensity or power corresponding to the applied electrical pulse). In some embodiments, system 301 includes one or more light sources 311 (such as a VCSEL (vertical-cavity surface-emitting laser), VCSEL array, point-source LED (light-emitting diode) array, and the like). In some embodiments, the light sources 311 emit light toward tissue volume 96 (which, as shown in FIG. 1A, may include overlying tissue 97 (e.g., skin, muscle and/or bone) and the tissue of interest 98). The scattered transmitted or reflected light returns and is detected by detectors 312, which generate electrical signals that are analyzed by signal processor 313. The signal processor 313 outputs one or more control signals which are used as described above for FIG. 1A. In other embodiments, pulse shapes other than square are used, e.g., triangular, saw-tooth (i.e., having either a fast rise time or a fast fall time), ramped up or down, or other suitable shapes.

FIG. 3B is a block diagram of neural-signal-capture system 302 that uses a plurality of simultaneous intensity-modulated-pulse light signals according to some embodiments of the present invention. In some embodiments, system 302 provides a plurality of electrical signals having the simultaneous intensity-modulated square-pulse shape as shown (amplitude in the vertical direction versus time in the horizontal direction), and applies the electrical pulse to one or more VCSEL sources 321 (which each generate a light pulse having a light intensity or power corresponding to the respective applied electrical pulse). In some embodiments, system 302 includes a plurality of light sources 321 (such as a VCSEL (vertical-cavity surface-emitting laser), VCSEL array, point-source LED (light-emitting diode) array, and the like). In some embodiments, the light sources 321 emit light toward tissue volume 96. The scattered transmitted or reflected light returns and is detected by detectors 322 that in some embodiments, include a plurality of intensity-frequency filters 324 (i.e., bandpass filters that pass only signals within the intensity-modulation frequencies used in the modulation of the original light pulse), each of which generate electrical signals that are analyzed by signal processor 323. In some embodiments, each one of a plurality of the intensity-modulated square-pulses has an overall envelope with a square (constant-intensity) shape that is modulated with a plurality of cycles of a higher-frequency modulation frequency. (In other embodiments, pulse envelopes other than square are used, e.g., triangular, saw-tooth, ramped up or down, or other suitable shapes.) In the embodiment shown, a 10-ns pulse is modulated with seven cycles of a 700 MHz cosine wave for the uppermost signal shown, a 10-ns pulse is modulated with eight cycles of a 800 MHz cosine wave for the upper-middle signal shown, a 10-ns pulse is modulated with nine cycles of a 900 MHz cosine wave for the lower-middle signal shown, or a 10-ns pulse is modulated with ten cycles of a 1000 MHz cosine wave for the lowermost signal shown. In some embodiments, each of the different-modulated-frequency pulses is emitted simultaneously, wherein each detector is followed by a plurality of parallel-wired frequency filters (e.g., one filter having a relatively narrow bandpass at 700 MHz, another filter having a relatively narrow bandpass at 800 MHz, another filter having a relatively narrow bandpass at 900 MHz, and another filter having a relatively narrow bandpass at 1000 MHz). In some embodiments, the different intensity-modulation-frequency pulses are each launched from a different VCSEL at spaced-apart locations. Thus, each of the filters is outputting a signal that came from only one of the VCSEL locations, allowing simultaneous triangulation to the neural activities being monitored. In some embodiments, the simultaneous emission of pulses having a plurality of different intensity-modulation frequencies, along with detectors each having a corresponding set of bandpass filters at the different intensity-modulation frequencies, allows faster repetition of the pulses (a higher pulse-repetition rate (PRR)) and thus greater data acquisition at the cost of the additional filtering circuits 324 and/or signal-processing circuits 323. In some embodiments, device 302 outputs a series of such parallel-in-time intensity-modulated sets of light pulses one after another, with the time gap between pulses being relatively short (e.g., as short as 10 ns or less in embodiments similar to the embodiment shown (which shows only a single set of substantially parallel-in-time (simultaneous) pulses), such that the PRR can be as high as 50 million pulses per second (MPPS) (with 10-ns pulses separated in time by 10-ns gaps) or more. In some embodiments, each of a plurality of VCSEL sources can each emit at a different wavelength (e.g., 680 nm, 750 nm and 830 nm) and each of a plurality of detectors includes a wavelength bandpass filter tuned to a corresponding different wavelength (e.g., 680 nm, 750 nm and 830 nm), such that simultaneous pulses at different light wavelengths from the plurality of different emitters can be each detected by a different detector, further increasing the data-capture capability. The signal processor 323 outputs one or more control signals, used as described above for FIG. 1A.

FIG. 3C is a block diagram of neural-signal-capture system 303 that uses a plurality of sequential intensity-modulated-pulse light signals according to some embodiments of the present invention. In some embodiments, system 303 provides a plurality of electrical signals having the sequentially-launched intensity-modulated square-pulse shape as shown (amplitude in the vertical direction versus time in the horizontal direction), and applies the electrical pulse to one or more VCSEL sources 331 (which generate a light pulse having a light intensity or power corresponding to the applied electrical pulse). In some embodiments, system 303 includes a plurality of more light sources 331 (such as a VCSEL (vertical-cavity surface-emitting laser), VCSEL array, point-source LED (light-emitting diode) array, and the like). In some embodiments, the light sources 331 emit light toward tissue volume 96. The scattered transmitted or reflected light returns and is detected by detectors 332, each of which generates electrical signals that are analyzed by signal processor 333. In some embodiments, each one of a plurality of the intensity-modulated square-pulses has an overall envelope with a square (constant-intensity) shape that is modulated with a plurality of cycles of a higher-frequency modulation frequency. In some embodiments, each pulse is modulated with the same frequency, while in other embodiments, each pulse is intensity modulated using a different frequency (for example, in the embodiment shown, modulated with seven cycles of a 700 MHz cosine wave, with eight cycles of a 800 MHz cosine wave, with nine cycles of a 900 MHz cosine wave for the lower-middle signal shown, or with ten cycles of a 1000 MHz cosine wave). In some embodiments, each of the different-modulated-frequency pulses is emitted sequentially, wherein each detector is followed by one or more frequency filters. In some embodiments, the different intensity-modulation pulses are each launched from a different VCSEL at spaced-apart locations. In some embodiments, the sequential emission of pulses having a single intensity modulation frequency, along with detectors each having a corresponding single bandpass filter at the given intensity-modulation frequencies, allows a lower-cost, simpler system (for a given pulse-repetition rate (PRR)) without the cost of the additional filtering circuits (such as 324 of FIG. 3B) and/or signal-processing circuits 333. In some embodiments, system 303 includes a plurality of light sources 331 (such as a VCSEL (vertical-cavity surface-emitting laser), VCSEL array, point-source LED (light-emitting diode) array, and the like). In some embodiments, the light sources 331 emit light toward tissue volume 96. The scattered transmitted or reflected light returns and is detected by detectors 332, each of which generates electrical signals that are analyzed by signal processor 333. The signal processor 333 outputs one or more control signals which are used as described above for FIG. 1A.

FIG. 3D is a block diagram of neural-signal-capture system 304 that uses a plurality of rigid-unit portions 305, each having a plurality of VCSELs and a plurality of circumferential detectors, that are interconnected using flex circuitry 342 according to some embodiments of the present invention. In some embodiments, the rigid-unit portions 305 each include a plurality of VCSELs (e.g., three in the embodiment shown, however a fewer or greater number of such light emitters are used in other embodiments), each arranged in the center of two rows of circumferentially arranged detectors. In some embodiments, neural-signal-capture system 304 is formed into a skull-surrounding cap that is placed against the scalp of the patient (person 89) and used to capture and determine the locations of neural activity in a plurality of areas of interest.

FIG. 3E is a plan-view block diagram of rigid unit 305 having a plurality of VCSELs and a plurality of circumferential detectors (detectors 363 arranged around a circumference) according to some embodiments of the present invention. In some embodiments, each such rigid unit 305 includes a plurality of VCSELs (e.g., three in the embodiment shown, however a fewer or greater number of such light emitters are used in other embodiments), each arranged in the center of one or more rows of circumferentially arranged detectors (e.g., two rows of four detectors each in the embodiment shown, however a fewer or greater number of such rows and/or detectors per row are used in other embodiments). In some embodiments, rigid unit 305 is fabricated as a single integrated circuit chip, while in other embodiments, a hybrid module is formed from a plurality of component chips. In some embodiments, VCSEL/detector portion 306 is configured to emit and detect light of a first wavelength (e.g., 680 nm), while VCSEL/detector portion 306′ is configured to emit and detect light of a second wavelength (e.g., 750 nm), and VCSEL/detector portion 306″ is configured to emit and detect light of a third wavelength (e.g., 830 nm). In other embodiments, other wavelengths are used. In some embodiments, a VCSEL control electronics and power-driver circuit 351 drives the one or more VCSEL/detector portions 306, 306′, and/or 306″. In some embodiments, the plurality of detectors in each of the plurality of rows allows detection of scattered light in each of a plurality of directions, and the plurality of rows allows detection of scattered light at different radii. In some embodiments, an array having a much larger number of light emitters and detectors (e.g., arrays of 8-by-8, or 64-by-64, or other grid sizes of such sets of VCSEL/detector portions 306, 306′, and 306″ are used, or similar grids of VCSEL/detector portions 306, 306′, or 306″ all of a single wavelength).

FIG. 3F is a cross-section-view block diagram of VCSEL/detector 306 having one VCSEL and a plurality of circumferential detectors (only one of which is shown here) according to some embodiments of the present invention. In some embodiments, a plurality of electrical contacts 361 (only one is shown here to simplify the drawing) provide electrical connections to the detector 363 and VCSEL active layer 365, a bottom-side (relative to this drawing) very-high-reflectivity mirror 366 and a top-side (relative to this drawing) partially transmissive and high-reflectivity mirror 364 provide laser feedback to active layer 365. In some embodiments, a suitable substrate material such as GaAs, GaN, sapphire or the like is used upon which to fabricate the other portions. In some embodiments, a focusing reflector and/or lens element 368 is used to output the light signal 369. In some embodiments, a wavelength-bandpass filter 362 limits the range of wavelengths that reach detector 363, in order to reduce background light (noise) detection and improve the signal/noise (S/N) ratio. In some embodiments, each detector 363 is sensitive for high-frequency (e.g., 50 MHz to 1 GHz or higher frequencies) bandwidth intensity-modulated light signals. That is, the wavelength bandwidth of filter 362 is narrow (e.g., wavelengths centered on the emission wavelength plus-or-minus 10 nm or less (or narrower in some embodiments, e.g., plus-or-minus 5 nm, or plus-or-minus 2 nm or plus-or-minus 1 nm)), while the frequency bandwidth of the detector is high (e.g., 50 MHz to 1 GHz or more (broader)).

FIG. 3G is a block diagram of neural-signal-capture system 307 that uses one or more intensity-modulated-pulse light signals and a plurality of detectors according to some embodiments of the present invention. In some embodiments, system 307 provides a modulation source that outputs a plurality of electrical signals having the simultaneous (or sequential) intensity-modulated square-pulse shape as shown in FIG. 3B (or FIG. 3C) above (which illustrate amplitude in the vertical direction versus time in the horizontal direction), and applies the electrical pulse to one or more VCSEL sources 371 (which each generate a light pulse having a light intensity or power corresponding to the respective applied electrical pulse). In some embodiments, system 307 includes a plurality of light sources 371 (such as a VCSEL (vertical-cavity surface-emitting laser), VCSEL array, point-source LED (light-emitting diode) array, and the like). In some embodiments, the light sources 371 emit light toward tissue volume 96. The scattered transmitted or reflected light returns and is detected by detectors 372 that in some embodiments, each of which generate electrical signals that are analyzed by signal processor 373.

In some embodiments, the present invention, using the various strategies for neural detection, uses component optical devices and electronic and/or software signal-processing technology that are assembled to form systems of the present invention. In some embodiments, the optical components include: an optical source having micro LED's (single channels or arrays), VCSELs (vertical-cavity surface-emitting laser arrays) (single channels or arrays) diode-driven solid-state lasers (SSLs) and/or other small, light-emitting substrates. Some embodiments include optical fibers coupled to the light emitter on one end and placed against or near the tissue of interest at the other end for light delivery. In some embodiments, the detector includes one or more small detectors that are matched to the wavelength and power characteristics of the expected or predicted signal when light from the optical source is applied to tissue or region of interest. In some embodiments, the source and detector are matched to one another for each channel, while in other embodiments, a single source is used in a configuration with numerous detector elements. Some embodiments use one or more optical fibers or other optical elements (such as lenses and the like) for light collection.

In some embodiments, the tissue of interest includes neural tissues. In some embodiments, the detected signal indicates the fluid or ion pressure and/or level of activity in a given region. The detected and/or recorded response is converted to meaningful data showing the intended body function. In some embodiments, this data is output as a signal whose intended use is to be sent as space-and-time-sensitive signals to drive the nerve stimulator within the prosthetic device.

When the tissue of interest is the human brain, some embodiments use devices for signal capture of neural activity in the brain, wherein these devices include: VCSEL or micro-LED array (if one source-detector for each functional channel) that is placed on the brain/cortex or the dura or the skull or the skin. The source will pulse or continuously apply light and the detector will sample at a defined rate. Information on the power density of the source and the light intensity collected at the detector and the morphology/geometry of the target tissue can be used to monitor neural activity in a spatially and temporally selective manner.

Movement of the brain relative to the emitter-detector probe depends on the location of the probe (whether the probe is inside the skull on the dura (which achieves greater stability and less movement), or outside the skull and/or scalp (which has more movement, but is less invasive and provides other advantages). Accordingly, some embodiments that use probes outside the scalp include remapping software that lets the user remap which emitters and/or detectors are used to detect particular neural patterns.

In some embodiments, the source includes a single array, a two-dimensional array (either in a flat (i.e., single plane, or a plurality of planes connected to one another using flexible (flex) circuitry), or along a curved surface such as entirely using a flex circuit), and/or a three-dimensional array (e.g., using a plurality of flex circuits) of light emitters such as VCSELs or light-emitting diodes (LEDs). In some embodiments, each channel includes a single detector, while in other embodiments, each channel includes a plurality of detectors.

In some embodiments, source-detector size and geometry is optimized to maximum light intensity collection (i.e., strong signal capture). Source or detector may have beam-shaping optics to only collect signals of a certain depth. The timing of on-off of a single source or channel can be used to locate the recorded response from a plurality of detectors (i.e., to provide higher contrast and higher resolution).

In some embodiments, the source/detector signal-capture system is passively placed over the tissue region of interest and embedded into the skull for stability. The system of the present invention can transmit through some bone or bone and skin. In some embodiments, portions or all of the system may be placed below the dura.

Nerve-potential detection and location-determining devices of the present invention for signal capture of neural activity in nerve or spinal cord, in some embodiments, include a cuff surrounding the spinal cord or a nerve or nerve bundle with source-detector pairs separated by 180 degrees, such that information regarding the signal (neural activity) is contained in the transmissive characteristics of the light from the source to the detector. By using many source-detector sets simultaneously, the position in three-dimensional (3-D) space of a given signal can be extrapolated by signal processing and sent to the prosthesis device.

Some embodiments include a cuff surrounding tissue with source-detector pairs adjacent to each other such that signal is contained within the reflective characteristics of the light. Position and beam-shaping optics will control depth of tissue probed (in addition to laser parameters used, like wavelength).

In some embodiments, these have a cylindrical geometry, such that a plurality of depths can be analyzed with the device fixed at a given tissue-surface position. The device may be positioned along any portion of the nervous system for signal capture.

The use of optical spectroscopy for detecting and determining the locations and time periods (the spatial and temporal characteristics) of neural activity provides unprecedented resolution (generally 10 to 50 times better than current techniques), is less sensitive to motion, and is very fast. Optical spectroscopy is also damage-free because the process is less invasive (outside the dura or skull), and the intensity is well below Food and Drug Administration (FDA) standards (the average power is less than 1 milliwatt (mW) through the skull). The penetration depth for optical spectroscopy is generally greater than 1 centimeter (cm) in the cortex and nerves.

Near-infrared spectroscopy (NIRS) is a specific type of spectroscopy used to detect neural activity. NIRS detects action potentials through fast-scattering changes in real time and is effective for a variety of wavelengths (this provides a plurality of source options). NIRS can run in a continuous wave (DC) or pulsed (AC modulation) mode and the latency is generally in the tens of milliseconds. The wavelength operation for NIRS generally varies from 690 nanometers (nm) to 830 nm, or in some embodiments, up to about 904 or 1200 nm, but the longer wavelengths are preferred because at shorter wavelengths (e.g., 690 nm), scattering is decreased and hemoglobin absorption is increased (thereby decreasing the signal-to-noise ratio), whereas at longer wavelengths (e.g., 830 nm), scattering is increased and hemoglobin absorption is decreased (thereby increasing signal-to-noise ratio). In some embodiments, the use of a plurality of detectors each detecting one of a plurality of different wavelengths in NIRS improves signal-to-noise ratio and contrast.

In some embodiments, the present invention provides an apparatus that performs time-resolved NIRS to measure neural activity. In some embodiments, this time-domain spectroscopy uses an optical pulse source (that emits pulses that have a duration of about 100 picosecond (ps)), since, in some embodiments, little background subtraction is required with such a short pulse (which provides increased signal-to-noise (↑ S/N) ratio). Some embodiments further include a detector unit that measures light intensity and “time of flight” from a plurality of detector sensors that are analyzed by a signal processor via a time point spread function. Some embodiments further include a plurality of detectors to reduce the influence of noise due to, e.g., superficial layers of tissue, and changes in tissue (i.e., the interfaces between tissue types having differing indices of refraction cause reflections, some of which, in some embodiments, are noise relative to the signal that is desired to be detected).

Some embodiments use optical-electrodes (optrodes) which conduct light signals and electrical signals to and/or from the tissue of interest (e.g., using an optical signal to stimulate a CNAP and detecting the resulting CNAP with the electrode, or vice versa). Some embodiments use an interface gel or other light-coupling enhancement between the light emitters and the patient's skin. Some embodiments use spatially resolved spectroscopy to reduce or cancel extraneous light noise.

In some embodiments, the present invention provides a method that includes calculating tissue optical properties using a diffusion-approximation analysis, either for calibration or for signal extraction.

Some embodiments detect a change in scattering to determine the intensity of a neural response (i.e., wherein higher-intensity neural activity (more neurons firing within a given period of time) indicate a higher intended force of muscle contraction), such that the force applied by the prosthetic device is based on the detected intensity of neural activity.

Some embodiments use time-of-flight determination and the source-detector separation to determine the exact location of neural activity (i.e., using an empirically calibrated brain activity map to determine which muscle the patient intended to move, and to thus control a prosthesis to effect that movement).

The present invention provides much quantitative information in a rapid manner and with high DR or data rate. The devices are highly sensitive and provide deep penetration.

On the other hand, in some embodiments, the instrumentation is large and when considered as a whole is commercially unavailable, and thus is developed using off-the-shelf components and parts. These are then changed to a commercially viable form suitable for economies-of-scale improvements to reduce cost. Also, some embodiments include a slow hemodynamic response present in signal.

Some embodiments use a phase-modulated NIRS to measure neural activity. In some such embodiments, the methodology used includes frequency-domain spectroscopy using Fourier-type frequency analysis. In some such embodiments, the emitted light signal pulse has been intensity modulated at 50 MHz to 1 GHz and has an optical power on the order of tens to 100 microwatts (μW). In some embodiments, the short pulse requires little background subtraction, resulting in increased signal/noise ratio (↑ S/N). In some embodiments, the detected light signal is measured in a manner that determines mean light intensity (e.g., measured as a DC amplitude), amplitude (e.g., measured as an AC amplitude), and phase of wave from each one of a plurality of detectors. In some embodiments, the time-of-flight is determined from the phase measurement. In some embodiments, a sequence of pulses each is modulated using a different frequency, wherein this scan through a plurality of frequencies allows adequate detection using fewer detectors (↓ #D's), while a single frequency can be used for the intensity-modulation frequency if the device is operated in spatially resolved spectroscopy (SRS) mode.

Some embodiments of the present invention use a plurality of detectors (a plurality of D's using a plurality of intensity-modulation frequencies and/or wavelengths) such that noise influence of various tissue features (e.g., superficial layers, changes in tissue, placement of optrodes, light coupling) is cancelled out.

Some embodiments simulate or calculate tissue optical properties with diffusion-approximation analysis, in order to calibrate the device relative to depth and spot location. For example, for each optical source, a calibration procedure determines which sensors are giving what signal in response to the patient desiring a particular movement or other activity.

In some embodiments, the DC signal is captured (in some embodiments, this is typically relatively large), and subtracted from the signal to eliminate noise (which significantly enhances the S/N ratio).

In some embodiments, the AC amplitude is captured and used to estimate the intensity of neural activity and this in turn controls a corresponding response (i.e., intended force of muscle contraction drives a corresponding prosthesis movement or motor-nerve stimulation closer to the patient's muscle to be controlled).

In some embodiments, the phase change in the detected signal is detected, and used with the given distance value (indicating source-detector separation) to determine the exact location of neural activity (e.g., a location on the brain, which location is mapped to three-space and used to determine which muscle the patient intended to move).

In some embodiments, the advantages of this technique include that it is technically relatively simple, there is rapid signal capture with a high S/N ratio. It is highly sensitive and has deep penetration. On the other hand, the instrumentation is relatively large; it uses relatively costly lasers and detectors, and complex signal processing.

In some embodiments, the present invention provides an apparatus that includes optical cellular arrays (e.g., a VCSEL array—vertical-cavity surface-emitting laser array). These have sources as small as 50 μm (microns), the pulses can have a duration (pulse width) as short as 100 ps (or shorter), or longer pulses with durations through continuous wave (CW) (wherein CW can have light emitted as long as power is applied). In some embodiments, the power is greater than 1 milliwatt (mW) per channel in NIR wavelengths. In some embodiments, a plurality of wavelengths is used (resulting in increased speed and increased signal/noise ratio).

In some embodiments, the present invention provides a device for reliable, precise signal capture, which generates one or more signals to drive a neuroprosthetic device.

Some embodiments develop a plurality of individual “channels” each having one or more sources, a plurality of detectors, and control electronics. Some embodiments empirically optimize source-detector (S-D) separation and geometry to increase contrast and the quality of the signal. In some embodiments, the present invention uses small NIR VCSELs with improved efficiency to reduce the electrical power consumption for implanted devices, as well as using highly sensitive, miniaturized detectors, and empirically optimized pulse characteristics (such as pulse duration, frequency of the intensity modulation, and wavelength(s)).

Some embodiments use a VCSEL array having a plurality of channels, wherein the channels are clustered with a plurality of channels per functional neural group (e.g., using a plurality of channels to distinguish intended movement of the index finger versus the middle finger). Some embodiments empirically optimize “channel” separation by changing the position of the source-detector unit, or by changing the mapping selections of the emitter and sensors while leaving the source-detector unit in the same one physical location, such that the light emission and detection pattern is modified (e.g., under software control) without moving the device.

In some embodiments, the present invention provides an apparatus that includes integrated, miniaturized electronics to reduce overall device size and to reduce power consumption.

In some embodiments, the present invention provides software using statistical-analysis algorithms and supporting software, which are run on suitable hardware (e.g., embedded high-speed, compact signal processors with high-reliability operating systems). The statistical-analysis algorithms output one or more signals based on the analysis of the detected signal's DC, AC and phase, as well as detector position, to determine location of neural activity. Some embodiments further include eliciting, receiving and using other user input as to the degree of bodily function desired. The resulting output signal(s) of the present invention provide high-sensitivity, high-specificity control to the interface with neuroprostheses.

In some embodiments, the present invention co-registers the placement of the implanted device with functional magnetic-resonance imaging (fMRI) during performance of functional tasks by the patient. Once implanted, the detected response is correlated with the degree and location of functional movement desired by the patient. The device is then calibrated using this information (which may change due to movement of the device) or a determination that other brain patterns can be better utilized to achieve the desired function can be used to remap the detection criteria for a particular desired output. In some embodiments, position accuracy is optimized using task-based analysis (having the patient attempt to perform various tasks, analyzing the signals that are captured), and then adjusting the signal processing to output the control signal that specifies that the prosthesis performs the function corresponding to the patient's intention. Other embodiments omit the fMRI and simply use the patient's expressed description of what was intended to map a particular detection pattern to a particular intended result.

In some embodiments, a muscle contraction is correlated to a detected and/or recorded nerve-activity-detection signal using statistical analysis, which provides high sensitivity and specificity.

An Appendix C is attached at the end of U.S. Provisional Patent Application 61/081,732 (Attorney Docket 5032.044PV1) filed on Jul. 17, 2008, titled “METHOD AND APPARATUS FOR NEURAL SIGNAL CAPTURE TO DRIVE NEUROPROSTHESES OR BODILY FUNCTION,” which is incorporated herein by reference in its entirety. That Appendix C contains additional information on the methods and apparatus for neural-signal capture to drive neuroprosthesis.

In some embodiments, the present invention provides an apparatus that includes at least one light source, the at least one light source configured to output a light pulse having a wavelength onto a volume of human tissue; at least one light detector configured to receive light reflected and transmitted by the volume of human tissue and to transmit an electrical signal, wherein the light reflected and transmitted by the volume of human tissue provides an indication of neural activity; a signal-processing unit operatively coupled to the at least one light detector and configured to receive the electrical signal from the at least one light detector. Some embodiments further include a stimulator unit operatively coupled to the signal-processing unit and configured to output a response signal to a prosthetic device. Some embodiments further include the prosthetic device.

This signal-processing unit correlates the electrical signal from the detector detecting of light to a particular location within the patient to quantify the neural signal (temporal characteristics, location (therefore function), and amplitude of the response (how many neurons are firing)). Also, in some embodiments, a portion of the signal processing removes motion artifacts due to the movement of the tissue volume of interest (e.g., the brain sloshing around in the skull cavity while the device is fixed to the skull will change the boundary conditions for the interpretation of the response).

In some embodiments, the present invention detects nerve or other tissue activities of one or more peripheral nerves and/or surrounding tissues (e.g., epineurium, perineurium, endoneurium), and/or spinal cord and/or surrounding cerebral spinal fluid and/or bone/cartilage.

In some embodiments of the apparatus, the at least one light source includes a vertical-cavity surface-emitting laser (VCSEL).

In some embodiments of the apparatus, the at least one light source includes a plurality of light sources, wherein the plurality of light sources includes a one-dimensional array of vertical-cavity surface-emitting lasers (VCSELs), and the at least one light detector includes a plurality of light detectors corresponding the plurality of light sources.

In some embodiments of the apparatus, at least one light source includes a plurality of light sources, wherein the plurality of light sources includes a two-dimensional array of vertical cavity surface emitting lasers (VCSELs), and the at least one light detector includes a plurality of light detectors corresponding the plurality of light sources.

In some embodiments of the apparatus, the at least one light source includes a micro-light-emitting diode (micro-LED).

In some embodiments of the apparatus, the at least one light source includes a plurality of light sources, wherein the plurality of light sources includes a one-dimensional array of micro-light-emitting diodes (micro-LEDs), and the at least one light detector includes a plurality of light detectors corresponding the plurality of light sources.

In some embodiments of the apparatus, the at least one light source includes a plurality of light sources, wherein the plurality of light sources includes a two-dimensional array of micro-light-emitting diodes (micro-LEDs), and wherein the at least one light detector includes a plurality of light detectors corresponding the plurality of light sources.

In some embodiments of the apparatus, the volume of human tissue further includes: neuronal tissue of the human brain; a dura layer located on the neuronal tissue of the human brain; a skull layer located on the dura layer; and a skin layer located on the skull layer. In some such embodiments, the light pulse traverses through the skin layer, the skull layer, and the dura layer before encountering the neuronal tissue of the human brain. In some embodiments, the light pulse traverses through the skull layer and the dura layer before encountering the neuronal tissue of the human brain. In some embodiments, the light pulse traverses through the dura layer before encountering the neuronal tissue of the human brain. In some embodiments, the at least one light source is embedded into the skull layer and the light pulse traverses through at least a portion of the skull layer and through the entire dura layer before encountering the neuronal tissue of the human brain.

In some embodiments of the apparatus, the volume of human tissue includes neuronal tissue of a human brain. In some embodiments of the apparatus, the volume of human tissue includes neuronal tissue of a human spinal cord and/or surrounding structures.

In some embodiments of the apparatus, the at least one light source includes a plurality of light sources and the at least one light detector includes a plurality of light detectors, wherein the plurality of light sources and the plurality of light detectors are arranged circumferentially around the volume of human tissue such that the plurality of lights sources alternates with the plurality of light detectors around the volume of human. In some embodiments of the apparatus, the at least one light source is located outside the skull of a human and interacts with tissue of the brain inside the skull of the human.

In some embodiments of the apparatus, the wavelength of the light pulse is between about 650 nm and about 850 nm. In some embodiments of the apparatus, the wavelength of the light pulse is between about 700 nm and about 825 nm. In some embodiments of the apparatus, the wavelength of the light pulse is between about 775 nm and about 825 nm. In some embodiments of the apparatus, the wavelength of the light pulse is between about 800 nm and about 850 nm.

In some embodiments, the present invention provides a method that includes outputting a light pulse having a wavelength onto a volume of human tissue such that the light pulse interacts with the volume of human tissue; detecting neural-signal activity by measuring a light signal resulting from the interaction of the light pulse with the volume of human tissue; transmitting an electrical signal based on the reflected and transmitted light signal; processing the electrical signal; and outputting a response signal to a prosthetic device based on the processing of the electrical signal to control an action by the prosthetic device.

In some embodiments of the method, the volume of human tissue includes brain tissue inside a human skull, and the outputting of the light pulse is done outside the human skull. In some embodiments of the method, the outputting a light pulse includes configuring a vertical-cavity surface-emitting laser (VCSEL) to emit light at a wavelength of about 675 nm to about 850 nm. In some embodiments of the method, the outputting a light pulse includes configuring a plurality of vertical-cavity surface-emitting lasers (VCSELs) to emit light at a wavelength of about 675 nm to about 850 nm. In some embodiments of the method, the outputting a light pulse includes configuring a micro-light-emitting diode (micro-LED) to emit light at a wavelength of about 675 nm to about 850 nm. In some embodiments of the method, the outputting a light pulse includes configuring a plurality of micro-light-emitting diodes (micro-LEDs) to emit light at a wavelength of about 675 nm to about 850 nm.

In some embodiments, the present invention provides a method that includes signal capture (detection) of neural activity using optical spectroscopy, and outputting a control signal based on the detected neural activity. In some embodiments, the neural activity includes neural activity of the central nervous system (i.e., the brain and/or spinal cord). In some embodiments, different geometry devices are used for the brain (e.g., detection of retro-reflection or angled scattering of one or more input optical pulses, wherein the devices have emitters and detectors that are all on one side of the tissue being observed), the spinal cord (e.g., detection of retro-reflection or angled scattering of one or more input optical pulses or of transmission of the light pulses wherein the devices have emitters and detectors that are surrounding the spinal-cord tissue being observed), and the peripheral nervous system (e.g., detection of retro-reflection or angled scattering of one or more input optical pulses or of transmission of the light pulses wherein the devices have emitters and detectors that are much closer to the small-diameter the peripheral nerves being observed). In some embodiments, the present invention provides a method that includes using of near-infrared spectroscopy (NIRS) and using time-domain and/or frequency-domain optical signal capture. In some embodiments, the present invention provides an apparatus that includes laser sources (such as semiconductor lasers) having rise and/or fall times on the order of ten (10) picoseconds in order to obtain spatial resolutions on the order of one (1) mm. In some embodiments, an emitter array that includes one or more vertical-cavity surface-emitting laser (VCSEL) arrays is used to emit pulses from a plurality of locations (e.g., a Cartesian grid) over an area of neural tissue to be observed.

In some embodiments, the emitter array selectively (e.g., under control of a microprocessor or other controller) emits light that has been amplitude-modulated (i.e., intensity modulated at, e.g., 50 MHz to 1 GHz and has an optical power on the order of, e.g., tens to 100 microwatts (μW)). In some embodiments, the present invention uses pulses that have a duration of about 100 picosecond (ps), since, in some embodiments, little background subtraction is required with such a short pulse (which provides increased signal-to-noise (↑ S/N) ratio).

In some embodiments, the present invention provides a method that includes measuring mean light intensity (DC) of the detected signal(s) as well as amplitude (AC), and phase of the detected waveform from a plurality of detectors. Some embodiments use the detected phase to determine the time-of-flight. Some embodiments scan through a plurality of intensity-modulation frequencies in order to reduce the number of detectors required. Some embodiments use a single frequency if they are operates in SRS (spatially resolved spectroscopy) mode.

In some embodiments, the emitted light pulses are all of a single wavelength but are amplitude modulated with a modulation frequency of between about 50 MHz and about 1 GHz or more, and the detectors are optionally wavelength-tuned or filtered to detect the emitted wavelength (e.g., the scattered light having the same wavelength as the emitted wavelength), wherein the neural activity changes the relative DC, AC amounts of the detected wavelength as well as the phase of the detected modulated light waveform. In some embodiments, the emitted light is also within an envelope of a pulse having a duration of about 1 nanosecond to about 1 microsecond. In other embodiments, the emitter array selectively (e.g., under control of a microprocessor or other controller) emits light pulses of a plurality of different wavelengths, and the detectors separately detect different wavelengths, in order to detect and differentiate between different nerve activities (e.g., triggering CNAP pulse versus cell recovery between CNAP pulses) and/or differentiate between activity at different spatial locations or depths. These approaches are termed “frequency-domain” detection herein because of the use of the intensity modulation (e.g., having the frequency between about 50 MHz and about 1 GHz or more) and the phase detection, which is used to determine time-of-flight. In some embodiments, such approaches need not precisely determine the time the envelope of detected pulses relative to the emitted pulses because the phase detection provides that function. In some embodiments, the device outputs a control signal that is operatively coupled to control a prosthetic device such as a motorized robotic arm and hand, or leg and foot.

In some embodiments, the emitter array selectively (e.g., under control of a microprocessor or other controller) emits light pulses, wherein the emitted light pulses have sharp rise and/or fall times and/or are very short (e.g., having rise, fall, or durations that are on the order of about 10 picoseconds) and optionally are all of a single wavelength, and the detectors are very fast (and are optionally wavelength-tuned or filtered to increase signal-to-noise (S/N) ratios) to detect and differentiate a plurality of different time-of-flight durations (e.g., the scattered light having the same wavelength as the emitted wavelength), wherein the neural activity changes the relative amounts of scattering or reflection of the emitted wavelength. In other embodiments, the emitter array selectively (e.g., under control of a microprocessor or other controller) emits light pulses from different locations, and the array of detectors separately detect and differentiate the different time-of-flight durations, in order to determine and differentiate between different nerve-activity spatial locations and/or depths. In some embodiments, a signal-processing operation is performed on a plurality of detected signals to determine the location of the neural activity. These approaches are termed “time-domain” detection herein. In some embodiments, the device outputs a control signal that is operatively coupled to control a prosthetic device such as a motorized robotic arm and hand, or leg and foot. In other embodiments, the control signal is operatively coupled to drive a closed-loop neuroprosthesis or neuro-modulation device.

In some embodiments, the emitter array selectively (e.g., under control of a microprocessor or other controller) emits light pulses, wherein the emitted light pulses are all of a single wavelength, and the detectors are wavelength-tuned or filtered to detect a plurality of different wavelengths (e.g., the scattered light having the same wavelength as the emitted wavelength and/or one or more longer wavelengths of fluoresced light), wherein the neural activity changes the relative amounts of the plurality of detected wavelengths. In other embodiments, the emitter array selectively (e.g., under control of a microprocessor or other controller) emits light pulses of different wavelengths, and the detectors separately detect different wavelengths, in order to detect and differentiate between different nerve activities (e.g., triggering CNAP pulse versus cell recovery between CNAP pulses) and/or differentiate between activity at different spatial locations or depths. Both of these approaches are termed “wavelength-domain” detection herein. In some embodiments, such approaches need not precisely determine the time the detected pulses relative to the emitted pulses-in some embodiments, the location of the nerve activity is very close to the device emitters and detectors such that the detection of light by a particular detector specifies that the neural activity was in the location adjacent to that detector without needing to determine a time (e.g., time-of-flight) duration. In some embodiments, the device outputs a control signal that is operatively coupled to control a prosthetic device such as a motorized robotic arm and hand, or leg and foot.

In some embodiments, the present invention provides a method that includes calibrating the device by associating a particular set of detected neural activity to a particular desired motor control, e.g., by empirically co-registering a functional image (what movement the patient desires to perform) to the sources and detectors that detect that brain activity (the set of spatially and temporally detected neural activities of various brain areas detected by one or more detectors using emitted light from one or more emitters, i.e., determining that these detectors are detecting neural activity resulting from the patient attempting middle finger movement in the upward direction).

In some embodiments of the method, the outputting of the light pulse includes intensity-modulating the light pulse at a frequency between about 50 MHz and about 1000 MHz. In some embodiments of the method, the light pulse traverses through the skin layer, the skull layer, and the dura layer and interacts with neuronal tissue of a human brain. In some embodiments of the method, the light pulse is intensity-modulated pulse at a frequency between about 50 MHz and about 1000 MHz. In some embodiments of the method, the intensity-modulated light pulse has a duration in a range of between about 10 ns and about 1000 ns.

In some embodiments of the method, the outputting of the light pulse is done from at least one light source is embedded into the skull layer and the light pulse traverses through at least a portion of the skull layer and through the entire dura layer and then interacts with neuronal tissue of a human brain.

In some embodiments, the present invention provides a method that includes outputting a light pulse having a wavelength onto a volume of human tissue such that the light pulse interacts with the volume of human tissue; detecting neural signal activity by measuring a resulting light signal from the interaction; transmitting an electrical signal based on the measured light signal; processing the electrical signal to generate a response signal; and outputting the response signal to a prosthetic device based on the processing of the electrical signal to effect an action by the prosthetic device.

In some embodiments of the prosthesis-control method, the outputting of the light pulse is done outside a skull of a human and the volume of animal tissue includes human brain tissue inside the skull of the human.

In some embodiments of the prosthesis-control method, the outputting of the light pulse includes emitting light at a wavelength of about 675 nm to about 850 nm from a vertical-cavity surface-emitting laser (VCSEL).

In some embodiments of the prosthesis-control method, the outputting of the light pulse includes emitting light at a wavelength between about 675 nm to about 850 nm from a micro-light-emitting diode (micro-LED).

In some embodiments of the prosthesis-control method, the light pulse traverses through the skin layer, the skull layer, and the dura layer and interacts with neuronal tissue of a human brain.

In some embodiments of the prosthesis-control method, the outputting of the light pulse includes outputting a substantially square light pulse having a duration between about 1 ps and about 10 ps.

In some embodiments of the prosthesis-control method, the outputting of the light pulse includes outputting a substantially square light pulse having a duration between about 10 ps and about 100 ps.

In some embodiments of the prosthesis-control method, the outputting of the light pulse includes intensity-modulating the light pulse at a frequency between about 50 MHz and about 1000 MHz. In some such embodiments, the intensity-modulated light pulse has a duration in a range of between about 10 ns and about 1000 ns.

In some embodiments of the prosthesis-control method, the outputting of the light pulse is done from at least one light source is embedded into the skull layer and the light pulse traverses through at least a portion of the skull layer and through the entire dura layer and then interacts with neuronal tissue of a human brain.

In some embodiments, the present invention provides a method that includes outputting a light pulse having a wavelength onto a volume of human tissue such that the light pulse interacts with the volume of human tissue; detecting neural signal activity by measuring a resulting light signal from the interaction; transmitting an electrical signal based on the measured light signal; processing the electrical signal to generate a response signal; and outputting the response signal to a display device based on the processing of the electrical signal to display a spatial pattern of neural activity that changes over time.

In some embodiments of the display method, the outputting of the light pulse is done outside a skull of a human and the volume of animal tissue includes human brain tissue inside the skull of the human.

In some embodiments of the display method, the outputting of the light pulse includes emitting light at a wavelength of about 675 nm to about 850 nm from a vertical-cavity surface-emitting laser (VCSEL). In other embodiments, the outputting of the light pulse includes emitting light at a wavelength between about 675 nm to about 850 nm from a micro-light-emitting diode (micro-LED).

In some embodiments of the display method, the light pulse traverses through the skin layer, the skull layer, and the dura layer and interacts with neuronal tissue of a human brain.

In some embodiments of the display method, the outputting of the light pulse includes outputting a substantially square light pulse having a duration between about 1 ps and about 10 ps. In other embodiments, the light pulse has a duration between about 10 ps and about 100 ps.

In some embodiments of the display method, the outputting of the light pulse includes intensity-modulating the light pulse at a frequency between about 50 MHz and about 1000 MHz. In some such embodiments, the intensity-modulated light pulse has a duration in a range of between about 10 ns and about 1000 ns.

In some embodiments of the display method, the outputting of the light pulse is done from at least one light source is embedded into the skull layer and the light pulse traverses through at least a portion of the skull layer and through a dura layer and then interacts with neuronal tissue of a human brain.

In some embodiments, the present invention provides an apparatus that includes means for outputting a light pulse having a wavelength onto a volume of human tissue such that the light pulse interacts with the volume of human tissue; means for detecting neural signal activity by measuring a resulting light signal from the interaction and for transmitting an electrical signal based on the measured light signal; means for processing the electrical signal to generate a response signal; and means for outputting the response signal to a prosthetic device based on the processing of the electrical signal to effect an action by the prosthetic device. Some embodiments further include the prosthetic device.

In some embodiments of this prosthetic apparatus, the means for outputting of the light pulse includes a vertical-cavity surface-emitting laser (VCSEL) that emits light at laser light at a wavelength of about 675 nm to about 850 nm.

In some embodiments of this prosthetic apparatus, the means for outputting of the light pulse includes means for intensity modulating the light pulse at a frequency between about 50 MHz and about 1000 MHz. In some embodiments of this prosthetic apparatus, the intensity-modulated light pulse has a duration in a range of between about 10 ns and about 1000 ns.

In some embodiments, the present invention provides an apparatus that includes means for outputting a light pulse having a wavelength onto a volume of human tissue such that the light pulse interacts with the volume of human tissue; means for detecting neural signal activity by measuring a resulting light signal from the interaction and for transmitting an electrical signal based on the measured light signal; means for processing the electrical signal to generate a response signal; and means for outputting the response signal to a display device based on the processing of the electrical signal to display a spatial pattern of neural activity that changes over time. In some embodiments of this display apparatus, the means for outputting the light pulse performs its operational function outside a skull of a human and the volume of animal tissue includes human brain tissue inside the skull of the human.

In some embodiments of this display apparatus, the means for outputting the light pulse includes a vertical-cavity surface-emitting laser (VCSEL) that emits laser light at a wavelength of about 675 nm to about 850 nm. In some embodiments of this display apparatus, the means for outputting the light pulse includes a micro-light-emitting diode (micro-LED) that emit light at a wavelength between about 675 nm to about 850 nm.

In some embodiments of this display apparatus, the means for outputting the light pulse includes means for intensity-modulating the light pulse at a frequency between about 50 MHz and about 1000 MHz. In some embodiments, the intensity-modulated light pulse has a duration in a range of between about 10 ns and about 1000 ns.

It is to be understood that the above description is intended to be illustrative, and not restrictive. Although numerous characteristics and advantages of various embodiments as described herein have been set forth in the foregoing description, together with details of the structure and function of various embodiments, many other embodiments and changes to details will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein,” respectively. Moreover, the terms “first,” “second,” and “third,” etc., are used merely as labels, and are not intended to impose numerical requirements on their objects.

What is claimed is:

1. An apparatus comprising:
at least one light source, the at least one light source configured to output a light pulse having a wavelength onto a volume of human tissue;
at least one light detector configured to receive light reflected and transmitted by the volume of human tissue and to transmit an electrical signal, wherein the light reflected and transmitted by the volume of human tissue provides an indication of neural activity; and
a signal-processing unit operatively coupled to the at least one light detector and configured to receive and signal-process the electrical signal from the at least one light detector and to output a signal based on the signal-processed electrical signal from the at least one light detector.

2. The apparatus of claim 1, wherein the at least one light source includes a vertical-cavity surface-emitting laser (VCSEL).

3. The apparatus of claim 1, wherein the at least one light source includes a plurality of light sources, wherein the plurality of light sources includes a one-dimensional array of vertical-cavity surface-emitting lasers (VCSELs), and wherein the at least one light detector includes a plurality of light detectors, one or more of the plurality of light detectors corresponding to each of the plurality of light sources.

4. The apparatus of claim 1, wherein the at least one light source includes a micro-light-emitting diode (micro-LED).

5. The apparatus of claim 1, wherein the light pulse traverses through the skin layer, the skull layer, and the dura layer before encountering the neuronal tissue of the human brain.

6. The apparatus of claim 1, wherein the at least one light source is embedded into the skull layer and the light pulse traverses through at least a portion of the skull layer and through the entire dura layer before encountering the neuronal tissue of the human brain.

7. The apparatus of claim 1, wherein the volume of human tissue includes neuronal tissue of a human brain.

8. The apparatus of claim 1, wherein the volume of human tissue includes neuronal tissue of a human spinal cord.

9. The apparatus of claim 1, wherein the at least one light source includes a plurality of light sources and the at least one light detector includes a plurality of light detectors, wherein the plurality of light sources and the plurality of light detectors are arranged circumferentially around the volume of human tissue such that the plurality of lights sources alternates with the plurality of light detectors around the volume of human.

10. The apparatus of claim 1, further comprising the prosthetic device, wherein the output unit is configured to output a response signal to a prosthetic device.

11. A method comprising:
outputting a light pulse having a wavelength onto a volume of human tissue such that the light pulse interacts with the volume of human tissue;
detecting neural signal activity by measuring a resulting light signal from the interaction;
transmitting an electrical signal based on the measured light signal;
processing the electrical signal to generate a response signal; and
outputting the response signal to a prosthetic device based on the processing of the electrical signal to effect an action by the prosthetic device.

12. The method of claim 11, wherein the outputting of the light pulse is done outside a skull of a human and the volume of animal tissue includes human brain tissue inside the skull of the human.

13. The method of claim 11, wherein the outputting of the light pulse includes emitting light at a wavelength of about 675 nm to about 850 nm from a vertical-cavity surface-emitting laser (VCSEL).

14. The method of claim 11, wherein the outputting of the light pulse includes emitting light at a wavelength between about 675 nm to about 850 nm from a micro-light-emitting diode (micro-LED).

15. The method of claim 11, wherein the light pulse traverses through the skin layer, the skull layer, and the dura layer and interacts with neuronal tissue of a human brain.

16. The method of claim 11, wherein the outputting of the light pulse includes outputting a substantially square light pulse having a duration between about 1 ps and about 10 ps.

17. The method of claim 11, wherein the outputting of the light pulse includes outputting a substantially square light pulse having a duration between about 10 ps and about 100 ps.

18. The method of claim 11, wherein the outputting of the light pulse includes intensity-modulating the light pulse at a frequency between about 50 MHz and about 1000 MHz.

19. The method of claim 18, wherein the intensity-modulated light pulse has a duration in a range of between about 10 ns and about 1000 ns.

20. The method of claim 11, wherein the outputting of the light pulse is done from at least one light source is embedded into the skull layer and the light pulse traverses through at least a portion of the skull layer and through the entire dura layer and then interacts with neuronal tissue of a human brain.

21. An apparatus comprising:
means for outputting a light pulse having a wavelength onto a volume of human tissue such that the light pulse interacts with the volume of human tissue;
means for detecting neural signal activity by measuring a resulting light signal from the interaction and for transmitting an electrical signal based on the measured light signal;
means for processing the electrical signal to generate a response signal; and
means for outputting the response signal to a prosthetic device based on the processing of the electrical signal to effect an action by the prosthetic device.

22. The apparatus of claim 21, further comprising the prosthetic device.

23. The apparatus of claim 21, wherein the means for outputting of the light pulse includes a vertical-cavity surface-emitting laser (VCSEL) that emits light at laser light at a wavelength of about 675 nm to about 850 nm.

24. The apparatus of claim 21, wherein the means for outputting of the light pulse includes means for intensity modulating the light pulse at a frequency between about 50 MHz and about 1000 MHz.

25. The apparatus of claim 24, wherein the intensity-modulated light pulse has a duration in a range of between about 10 ns and about 1000 ns.
